(19) United States
(12) Patent Application Publication
(10) Pub. No.: US 2016/0251611 A1
(43) Pub. Date: Sep. 1, 2016

(54) A PROCESS FOR GROWING A MICROBIAL ORGANISM

(71) Applicant: BIOCHEMTEX S.P.A., Tortona (Alessandria) (IT)

(72) Inventors: Piero OTTONELLO, Milano (IT); Simone FERRERO, Tortona (IT); Paolo TORRE, Arenzano (IT); Stefano PARAVISI, Tortona (IT); Chiara PREFUMO, Genova (IT); Pietro PASTORINO, Campo Ligure (IT); Alessia FICALBI, San Salvatore Monferrato (IT)

(73) Assignee: Biochemtex S.p.A., Tortona (IT)

(21) Appl. No.: 15/032,659

(22) PCT Filed: Oct. 31, 2014
(86) PCT No.: PCT/EP2014/002925

§ 371 Date: Apr. 28, 2016

(30) Foreign Application Priority Data
Oct. 31, 2013 (IT) TO 2013 A 000888

(51) Int. Cl. C12N 1/22; C12N 1/16

(52) U.S. Cl. CPC C12N 1/22; C12N 1/16

(57) ABSTRACT

An method for growing a microbial organism, comprising the cultivation of the microbial organism in the presence of a hydrolyzed composition obtained from a thermally treated ligno-cellulosic biomass. The treatment preferably comprises a fiber shives reduction step. The hydrolyzed composition has very few inhibitor compounds and the microbial organism feed with the hydrolyzed composition grows in a short time with a high duplication factor.

### BACKGROUND

The conversion of sugars to useful biochemicals by means of living microbial organisms is used since many thousands of years. In particular yeasts, being able to ferment sugars to alcohols and carbon dioxide, are the basic component in bakery and alcoholic beverages production and they are the most used microbial organisms.

More recently, microbial organisms found a new set of applications in the conversion processes of ligno-cellulosic feedstocks to biofuels such as ethanol, and to organic compounds, with the aim to replace oil and other fossil sources.

The microbial organisms usually metabolize simple sugars, which are monomeric sugars and optionally dimeric sugars in the case of yeasts, thereby the need to hydrolyze the ligno-cellulosic feedstock for converting polymeric and oligomeric sugars to simple sugars. For increasing the accessibility to cellulose and hemicellulose and the hydrolysis effectively occurring, ligno-cellulosic feedstocks are usually subjected to a pre-treatment. Unfortunately, pre-treatments and hydrolysis produce ligno-cellulosic feedstock degradation products such as acetic acid, formic acid, furfural and hydroxymethylfurfural (5HMF) which have an inhibitory effect on the activity of many microbial organisms. Moreover, acid pre-treatments and hydrolysis processes are conducted in the presence of an inorganic or organic acids.

During cultivation, simple sugars may be converted to new microbial organism biomass and/or to organic compounds, depending on cultivation parameters such as aerobic condition and sugar concentration in the cultivation environment.

For cost consideration, microbial organisms are subjected to a growth phase for increasing the microbial organisms biomass. During growth in a batch configuration, microbial organisms reproduce at an exponential rate, after which the biomass of the microbial organism remains approximately constant. Exponential growth is usually preceded by a lag-phase, during which the microbial organisms adapt to the cultivation medium and there is no—or very limited—cell reproduction. Inhibitors usually have the effect of increasing the lag-phase and reducing biomass yield.

Typically, on a lab scale the growth is conducted by introducing synthetic monomeric sugars as carbon source and other media as nitrogen source in the cultivation environment. This solution is expensive on industrial scale, where monomeric sugars are supplied to the cultivation environment typically in the form of beet and sugar cane molasses.

The use of hydrolyzates of ligno-cellulosic feedstocks is of interest for further reducing the cost of microbial organism growth at industrial scale, provided that effects of inhibitors are reduced.

In Joao R. M. Almeida et al., “Screening of Saccharomyces cerevisiae strains with respect to anaerobic growth in non-detoxified lignocellulose hydrolysate”, Bioresource Technology, 100 (2009), p. 3674-367′7, the authors present the anaerobic growth of 12 yeast strains fed with mixtures of synthetic glucose and three different ligno-cellulosic hydrolyzates, concluding that yeast growth is supported till a hydrolyzate concentration in the range of 50% to 70%, depending on the hydrolyzate. Moreover, lag-phases are in the range of 15 h to 35 h.

As pointed out in T. Brandberg et al., “The fermentation performance of nine strains of Saccharomyces cerevisiae in batch and fed-batch cultures in dilute-acid wood hydrolysate”, Journal of Bioscience and Bioengineering, Vol. 98 (2004), No. 2, p. 122-125, a number of different strategies have been suggested to overcome the effect of inhibitors. Treatment with alkali generally improves the fermentability of the hydrolyzate. Other detoxification methods include treatment with laccase, sulphite, and evaporation. All such detoxification methods will add to the cost of the process.

There is therefore the need to develop an inexpensive method for growing a microbial organism by using hydrolyzates of ligno-cellulosic feedstocks containing low inhibiting effects.

### BRIEF DESCRIPTION OF THE INVENTION

It is disclosed a process for growing a microbial organism comprising the steps of:
a. Thermally treating a ligno-cellulosic biomass feedstock to create a thermally treated ligno-cellulosic biomass, said thermally treated ligno-cellulosic biomass comprising xylans, glucans and lignin;
b. dispersing an amount of the thermally treated ligno-cellulosic biomass into an amount of a carrier liquid to create a low viscosity slurry;
c. contacting the low viscosity slurry with an enzyme under hydrolysis conditions of a carbohydrate component of the low viscosity slurry, to produce a hydrolyzed composition comprising simple sugar or sugars derived from the xylans and glucans of the thermally treated biomass, wherein the simple sugar or sugars can be metabolized by the microbial organism;
d. cultivating the microbial organism in a cultivation environment comprising at least a portion of the hydrolyzed mixture under conditions and for a cultivation time sufficient to grow the microbial organism.

It is also disclosed that the thermally treated ligno-cellulosic biomass is in physical forms of at least fibres, fines and fiber shives, wherein: the fibres each have a width of 75 μm or less, and a fibre length greater than or equal to 200 μm; the fines each have a width of 75 μm or less, and a fine length less than 200 μm; the fiber shives each have a shive width greater than 75 μm with a first portion of the fiber shives each having a shive length less than 737 μm and a second portion of the fiber shives each having a shive length greater than or equal to 737 μm.

The process may further comprise the step of reducing the fiber shives of the thermally treated biomass, wherein the percent area of fiber shives having a shive length greater than or equal to 737 μm relative to the total area of fiber shives, fibres and fines of the thermally treated ligno-cellulosic biomass after fiber shives reduction is less than the percent area of fiber shives having a shive length greater than or equal to 737 μm relative to the total area of fiber shives, fibres and fines of the thermally treated ligno-cellulosic biomass before fiber shives reduction, wherein the total area of fiber shives, fibres and fines is measured by automated optical analysis.

It is further disclosed that a part of the fiber shives reduction may be done by separating at least a portion of the fiber shives having a shive length greater than or equal to 737 μm from the thermally treated ligno-cellulosic biomass.

It is also disclosed that part of the fiber shives reduction may be done by converting at least a portion of the fiber shives having a shive length greater than or equal to 737 μm in the thermally treated ligno-cellulosic biomass to fibres or fines.

It is further disclosed that at least a part of the fiber shives reduction step may be done by applying a work in a form of mechanical forces to the thermally treated ligno-cellulosic biomass, and all the work done by all the forms of mechanical forces on the thermally treated ligno-cellulosic biomass is less than 500 Wh/Kg per kg of the thermally treated ligno-cellulosic biomass on a dry basis.

It is also disclosed that all the work done by all the forms of mechanical forces on the thermally treated ligno-cellulosic biomass may be less than a value selected from the group consisting of 400 Wh/Kg, 300 Wh/Kg, 200 Wh/Kg, 100 Wh/Kg, per kg of the thermally treated ligno-cellulosic biomass on a dry basis.

It is further disclosed that the percent area of the fiber shives having a shive length greater than or equal to 737 μm relative to the total area of fiber shives, fibres and fines of the thermally treated ligno-cellulosic biomass after fiber shives reduction may be less than a value selected from the group consisting of 1%, 0.5%, 0.25%, 0.2% and 0.1%.

It is also disclosed that wherein the low viscosity slurry may have a viscosity less than a value selected from the group consisting of 0.1 Pa s, 0.3 Pa s, 0.5 Pa s, 0.7 Pa s, 0.9 Pa s, 1.0 Pa s, 1.5 Pa s, 2.0 Pa s, 2.5 Pa s, 3.0 Pa s, 4 Pa s, 5 Pa s, 7 Pa s, 9 Pa s, 10 Pa s, wherein the viscosity is measured at 25° C., at a shear rate of 10 s-1 and at a dry matter of 7% by weight.

It is further disclosed that the dry matter of the low viscosity slurry by weight may be higher than a value selected from the group consisting of 5%, 7%, 8%, 10%, 12%, 15%, 18%, 20%, 25%, 30%, 35%, 40%.

It is also disclosed that the amounts and types of respective ionic groups in the low viscosity slurry may be not greater than the amounts and types of the respective ionic groups present in the feedstock or formed in the thermal pre-treatment.

It is further disclosed that the amounts and types of respective ionic groups in the low viscosity slurry may be not greater than a value selected from the group consisting of 20%, 15%, 10%, 5%, 3%, 2%, 1% of the amounts and types of the respective ionic groups present in the feedstock or formed in the thermal pre-treatment.

It is also disclosed that the ionic groups may be derived from the group consisting of: mineral acids, organic acids and bases.

It is further disclosed that the thermal treatment of the ligno-cellulosic biomass feedstock comprises the step of steam exploding the ligno-cellulosic biomass feedstock to create the thermally pre-treated ligno-cellulosic biomass.

It is further disclosed that the steam explosion step may be preceded by the steps of:
a) Soaking the ligno-cellulosic biomass feedstock in vapor or liquid water or mixture thereof in the temperature range of 100 to 210° C. for 1 minute to 24 hours to create a soaked ligno-cellulosic biomass feedstock containing a solid content and a liquid content;
b) Separating at least a portion of the liquid content from the soaked ligno-cellulosic biomass feedstock to create a solid stream and a liquid stream, wherein the solid stream comprises the ligno-cellulosic biomass feedstock, which has been soaked.

It is also disclosed that the carrier liquid may further comprise at least a portion of the liquid stream.

It is further disclosed that the conversion of the ligno-cellulosic biomass feedstock to the low viscosity slurry may be conducted without the addition of a hydrolysis catalyst.

It is also disclosed that the hydrolyzed composition comprises acetic acid and the ratio of the amount of acetic acid to the total amount of simple sugar or sugars may be less than a value selected from the group consisting 0.15, 0.10, 0.05, 0.02. and 0.01.

It is further disclosed that the hydrolyzed composition may comprise furfural and the ratio of the amount of furfural to the total amount of simple sugar or sugars in the hydrolyzed composition is less than a value selected from the group consisting of 0.01, 0.005, 0.001, 0.0005, and 0.0003.

It is also disclosed that the hydrolyzed composition may comprise 5HMF and the ratio of the amount of 5HMF to the total amount of the simple sugar or sugars in the hydrolyzed composition is less than a value selected from the group consisting of 0.02, 0.01, 0.005, 0.001, and 0.0005.

It is further disclosed that the cultivation of the microbial organism may be done without added simple sugar or sugars to the cultivation environment.

It is also disclosed that the cultivation of the microbial organism may be done with added simple sugar or sugars, and the percent ratio of the amount of added simple sugar or sugars to the total amount of simple sugar or sugars of the hydrolyzed composition is less than a value selected from the group consisting of 30%, 20%, 10%, 5.0%, and 2.0%.

It is further disclosed that the cultivation time may be less than a value selected from the group consisting of: 36 hours, 24 hours, 18 hours, 12 hours and 6 hours.

It is also disclosed that the cultivation of the microbial organism may be performed in aerobic condition at an air flow which is less than a value selected from the group consisting of 1VVm, 10 VVh, 5VVh, 1VVh, 0.5VVh, 0.1 VVh, and 0.05VVh.

It is further disclosed that the microbial organism may be a non-naturally occurring microbial organism.

It is also disclosed that the microbial organism is a yeast and that the yeast may be selected from the group consisting of Saccharomyces, Zygosaccharomyces, Candida, Hansenula, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, and Schwanniomyces.

It is further disclosed that the microbial organism may be a bacterium.

### BRIEF DESCRIPTION OF FIGURES

FIG. 1 is the screw design of the twin screw extruder used in the experiments.

FIG. 2 depicts the glucans accessibility of thermally treated ligno-cellulosic biomass before and after fiber shives reduction at various severity factors of thermal treatment.

FIG. 3 depicts the glucose and xylose recovery of thermally treated ligno-cellulosic biomass before and after fiber shives reduction at various severity factors of thermal treatment.

FIG. 4 is fibres and fines distribution of thermally treated ligno-cellulosic biomass before and after fiber shives reduction at two severity factors of thermal treatment.

FIG. 5 is the fiber shives distribution of thermally treated biomass before shives reduction and the thermally treated biomass after shives reduction at two severity factors of thermal treatment.

FIG. 6 is the fiber shives content of thermally treated ligno-cellulosic biomass before and after fiber shives reduction as a function of the severity factor of thermal treatment.

FIG. 7 plots the torque of slurries of various experimental runs at different dry matter contents in the slurry.

FIG. 8 plots the torque of slurries made from 18% dry matter content of the thermally treated ligno-cellulosic biomass before and after fiber shives reduction as a function of the severity factor of thermal treatment.

FIG. 9 plots the saturation humidity of thermally treated ligno-cellulosic biomass before and after fiber shives reduction at different severity factors of thermal treatment.

FIG. 10 plots the torque measurement versus time of the thermally treated ligno-cellulosic biomass before and after fiber shives reduction.

FIG. 11 plots the viscosity of slurries of the thermally treated biomass after fiber shives reduction at different amounts in water.

FIG. 12 plots the viscosity of slurries of thermally treated ligno-cellulosic biomass before and after fiber shives reduction at different dry matter contents of the slurry.

FIG. 13 is a graph of the yeast amount concentration, ethanol concentration and sucrose concentration according to a comparative example.

FIG. 14 is a graph of the yeast amount concentration, ethanol concentration and sucrose concentration according to a working example of the invention.

### DETAILED DESCRIPTION

It is disclosed a method for growing a microbial organism, comprising the cultivation of the microbial organism in the presence of a hydrolyzed composition obtained from a ligno-cellulosic feedstock.

In the context of the present disclosure, by “growing a microbial organism”, or “microbial organism growth”, or “producing a microbial organism” it is meant the process of increasing the microbial organism amount, or microbial organism biomass, obtained by feeding an initial microbial organism amount, or inoculum or inoculated culture, with a carbon source and other nutrients in suitable conditions. The increase of the microbial organism biomass may occur by increasing the number of microbial organism cells or by increasing the size (weight) of the single cells, or both.

A microbial organism grows under specific cultural conditions. When the microbial organism is introduced into the cultivation environment, initially growth does not occur. This period is referred to as the lag phase and may be considered a period of adaptation. During the next phase referred to as the “exponential phase” the growth rate of the microbial organism gradually increases. After a period of maximum growth the rate ceases and the culture enters stationary phase. After a further period of time the culture enters the death phase and the number of viable cells of the microbial organism declines.

Inventors discovered an improved process for growing a microbial organism with respect to the processes well established in the art for growing a microbial organism. The improvement may be measured by means of the standard parameters used for evaluating the growth of a microbial organism, such as the lag-phase and the duplication factor at a fixed time.

The lag-phase is the time needed by the microbial organism to adapt to the cultivation environment and it ends with the first duplication of the initial microbial organism amount. A practical definition of lag-phase is the time needed for first duplication, i.e. the time needed for doubling the initial microbial organism amount.

The duplication factor at a reference time is the ratio of the amount of the microbial organism present in the cultivation environment at the reference time to the initial amount of the microbial organism. Reference time usually corresponds to approximately the end of the exponential growth phase.

From an industrial point of view; lag-phase should be as short as possible and duplication factor should be as high as possible. The disclosed method for growing a microbial organism has shorter lag-phase and higher duplication factors than other microbial organism growth methods. This is particularly important in the case of yeast growth.

Another important parameter is the cost of the carbon source. The hydrolyzed composition prepared according the disclosed treatment is a carbon source which is less expensive than synthetic and molasse derived carbon sources.

Inventors discovered a process for growing a microorganism which comprises a thermal treatment of a ligno-cellulosic biomass to produce a thermally treated ligno-cellulosic biomass which is in the physical form of fibres, fines and fiber shives. Preferably the thermal treatment is conducted at a low severity, more preferably without the use of added acids, or bases, and the thermally treated ligno-cellulosic biomass contains no or very few inhibitory compounds of the microorganism growth, such as furfural, acetic acid and 5HMF. The thermally treated ligno-cellulosic biomass is then subjected to a fiber shives reduction step, as explained in details in this specification, which increases the sugar enzymatic accessibility without producing inhibitory compounds. Thereby, by hydrolyzing some carbohydrates of the thermally treated ligno-cellulosic biomass after fiber shives reduction, it is obtained a hydrolyzed composition comprising simple sugars that the microbial organism may use as a carbon source, or metabolize, and wherein the hydrolyzed composition comprises very few inhibitory compounds which is particularly suitable for growing, or propagating, a microbial organism.

It is known in the paper and pulp industry that ligno-cellulosic biomass feedstocks are characterized by the content of its particles classified into fibres, fines and fiber shives. Fibres are measured on the basis of their 2 dimensional profile with fibres having a width of 75 μm or less, and a fibre length greater than or equal to 200 μm. Fines are those particles having a width of 75 μm or less, and a fines length less than 200 μm. Geometrically, one can think of a fine as a fibre which has been cut in length. Fiber shives have a shive width greater than 75 μm and can be any length. For the purpose of this specification the shive length can be categorized with a first portion of the fiber shives having a shive length less than 737 μm and a second portion of the fiber shives having a shive length in the range of greater than or equal to 737 μm. Because the width and length describe high aspect ratio particles, the width is less than the length, except in the special case of the circle or square. In the special case when the length and width equal each other the practitioner selects one measurement as the length and arbitrarily therefore, the other measurement as the width.

The 737 μm is selected on the basis of classification of the particle distribution determined by the instrument used in the experiments which gave rise to the disclosed discovery. The sizes of the particles were grouped, with one of the groups having a range of 737-1138 μm. The next group had 1138 as its minimum size. From these groups the graphs were made in figures and determinations made about the effective ranges needed to practice the discovery.

Dimensions of Common NonWood Fibers cited in the Kirk-Othmer Encyclopedia of Chemical Technology, fifth edition, are

| | Mean Length, | Mean Diameter, | |
|---|---|---|---|
| Fibre Source | μm | μm | L/D ratio |
| Rice straw | 1410 | 8 | 175 |
| Wheat straw | 1480 | 13 | 110 |
| Corn stalk | 1260 | 16 | 80 |
| Cotton stalk | 860 | 19 | 45 |
| Cotton liners | 3500 | 21 | 165 |
| Sugarcane bagasse | 1700 | 20 | 85 |
| Hemp | 20000 | 22 | 1000 |
| Kenaf bast | 2740 | 20 | 135 |
| Kenaf core | 600 | 30 | 20 |
| Seed flax | 27000 | 16 | 1250 |
| Bamboo | 2700 | 14 | 190 |
| Papyrus | 1500 | 12 | 125 |
| Softwood | 3000 | 30 | 100 |
| Hardwood | 1250 | 25 | 50 |

As evident, the average fibre width, as previously defined, is less than or equal to 75 μm.

It is generally viewed that the fiber shives are not a single fibre having the width greater than 75 μm, but bundle of fibres or fibre tangles which combined exhibit a width greater than 75 μm.

This invention is based upon the discovery it is the fiber shives in thermally treated ligno-cellulosic biomass which are responsible for the long enzymatic hydrolysis times, high initial viscosity of slurries from the thermally treated ligno-cellulosic biomass, and the lowered glucose recoveries and yields. This specification demonstrates that by reducing the amount (percentage) of the fiber shives in the thermally treated ligno-cellulosic biomass, the viscosity of the material in a slurry drops dramatically, and there is a significant improvement in sugar yields and recovery during fermentation.

The ability to characterize and fines, fibres and fiber shives is well known in the art and the subject of many industrial standards such as those found in the fiber characterization standards used for all the fiber characterization work in this specification.

Because fiber shives are bundles of fibres, they can be reduced in many ways. First, at least a part of the fiber shives can be removed or separated from the thermally treated ligno-cellulosic biomass. Separation techniques of fiber shives from fibres and fines is well known in the art of natural fibres (e.g. cotton, flax, and others) and also in the paper and pulp industry. Non-limiting examples are the cotton gin and wool carding apparata. Again, not limiting, the separation can occur by bulk density separation, a vibrating bed where the fiber shives separate from the fines and fibres, air elutriation, or even screening, sieving or cyclones. After separation, the fiber shives can be further processing into fibres or fines, and recombined with the thermally treated ligno-cellulosic biomass or re-fed into the thermal treatment process.

The fiber shives can also be reduced by converting them to another form. One method of converting the fiber shives is to apply mechanical forces to the thermally treated ligno-cellulosic biomass to convert the fiber shives to fibres and/or fines. An important consideration is that the difference between a fine and a fibre is the length, as both have a width of less than or equal to 75 μm. The application of mechanical forces to thermally treated ligno-cellulosic biomass is practiced in the art, but always under the belief that the fibres (less than or equal to 75 μm width) must be acted upon. By focusing the application of the mechanical forces upon the fiber shives which are bundles of fibres >75 μm, the amount of work needed is to obtain the benefits mentioned earlier is significantly less than prior art disclosures.

The reason for this reduced work requirement is analogized to yarn which is twisted fibres. It does not take much energy to pull apart a ball of tangled yarn, but it takes much more energy to actually destroy and pull apart the twisted yarn fibre.

The start of the process is the feedstock of thermally treated ligno-cellulosic biomass feedstock. The type of ligno-cellulosic biomass feedstock for the thermal treatment is covered in the feedstock selection section.

In typical conversion of ligno-cellulosic biomass feedstock to ethanol, the ligno-cellulosic biomass is thermally treated prior to enzymatic hydrolysis. Oftentimes this thermal treatment will include acids or bases to increase the liquefaction rate and reduce the hydrolysis time. In many cases the thermal pretreatment includes a steam explosion step.

The thermal treatment is measured by a severity factor which is a function the time and temperature of the thermal treatment. A preferred thermal treatment is described in the thermal treatment section of this specification.

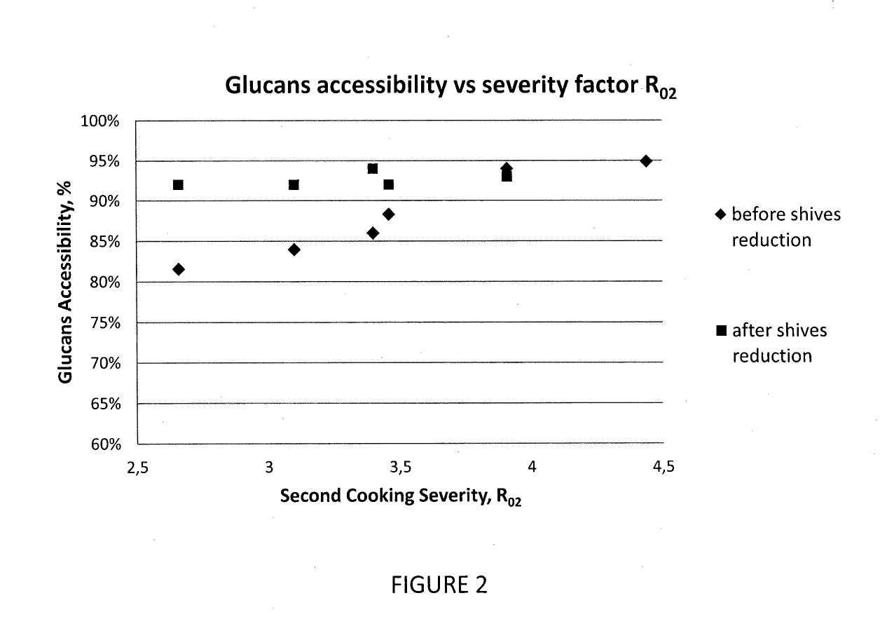

The more time of heat exposure, the more the severe the treatment. The higher the temperature of exposure, the more the severe the treatment. The details of calculating the severity factor for this invention are described later. Steam explosion severity factor (R₀₂) is taken as the reference severity factor. However, conventional wisdom holds that the more severe the treatment, the more surface area and cells of the ligno-cellulosic biomass are exposed to enzymes for hydrolysis or further treatment. This is demonstrated in FIG. 2, showing that the glucans become more accessible as the severity factor increases.

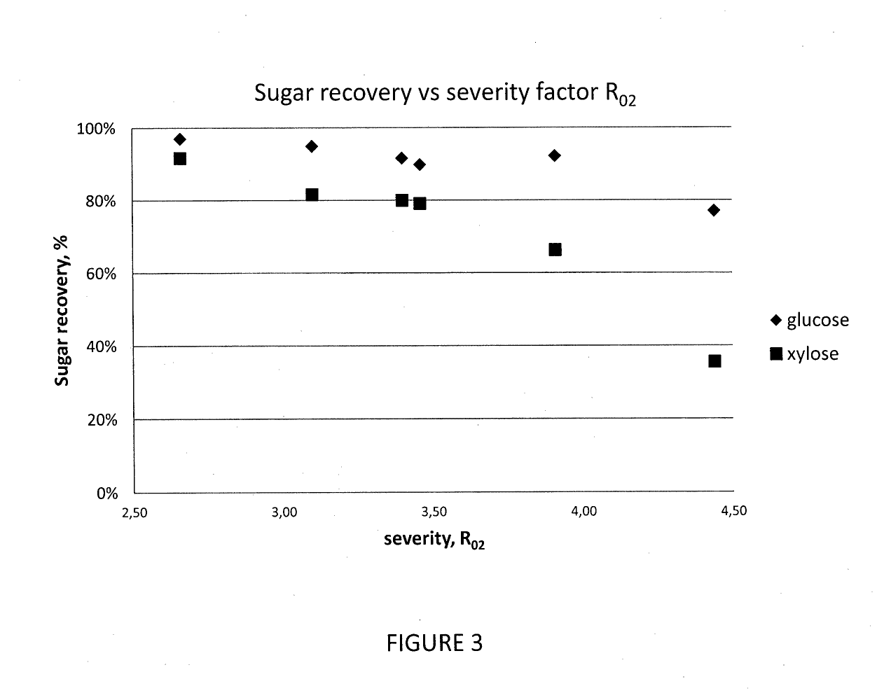

However, as demonstrated in FIG. 3, the amount of glucose and xylose that may be recovered relative to the amount present before the thermal treatment declines at higher severity factors. It is believed that the higher temperature converts or otherwise destroys the sugars. Thus, while the sugars existing in the thermally treated ligno-cellulosic biomass become more available, less sugars exist after severe thermal treatment because the severe temperature/time converts them to sugars degradation products, such as furfural and HMF.

Taking for example, FIG. 3, the points at severity factor 2.66, 97% of the glucose is present after the thermal treatment. In contrast, at a severity factor of 4.44 only 77% is recoverable, or alternatively 23% is destroyed. For xylose, almost 64% is destroyed. However, looking at FIG. 2, for the severity factor of 2.66, only 82% of the glucans are accessible or able to be converted to glucose. Thus, while 97% of the starting amount still exists, only 82% of that can be enzymatically converted. Looking at FIG. 2, severity factor 4.44, 95% of the glucans are accessible but remember from FIG. 3, that only 82% of the starting amount of glucans remains.

What has been discovered is that these inaccessible glucans reside in the fiber shives. When the biomass is processed it is often reduced to width and length that conform to fibres—high aspect ratio as defined in the standard. Usually the thermal treatment of the ligno-cellulosic biomass will create a thermally treated ligno-cellulosic biomass in the physical forms of at least fibres, fines and fiber shives. These physical forms are well known according the definitions described earlier.

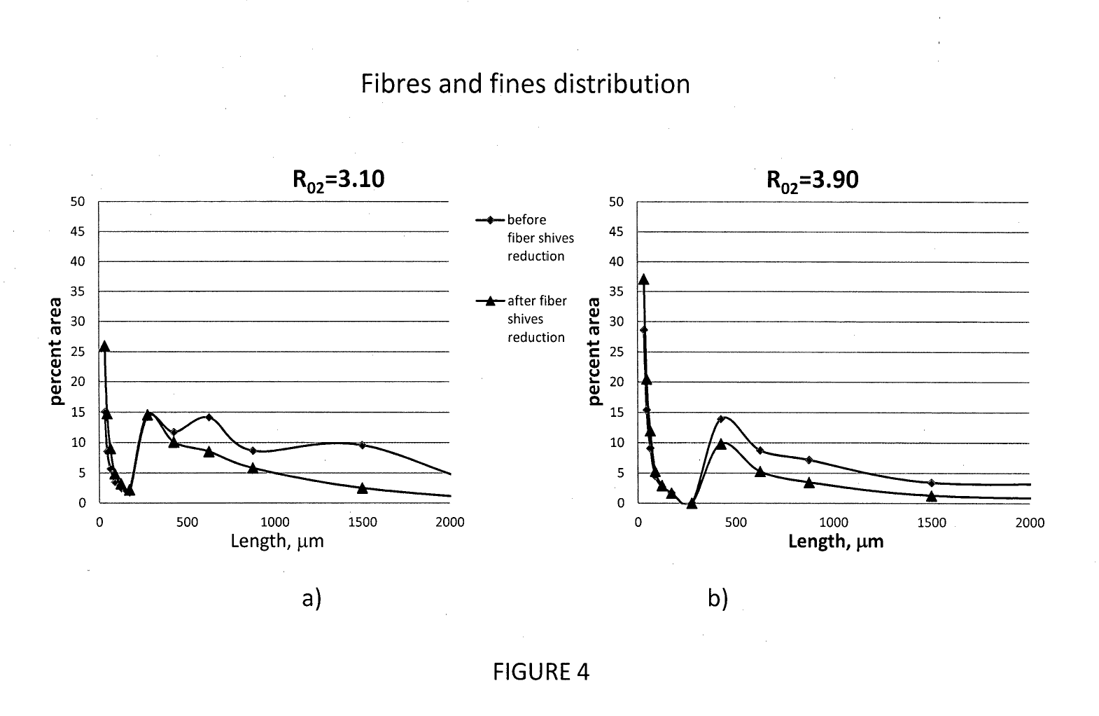

The fines and fibres (not shives) distribution of thermally treated ligno-cellulosic biomass is shown in FIG. 4. FIG. 4a) shows the percent area of each length class relative to the total area of fines, fibres and fiber shives for the severity factor R₀₂ of 3.1. When the severity factor is increased to 3.91, (FIG. 4b), it is evident that the percent area of fines has increased (particles of length <200 μm) and the percent area of fibres longer than or equal to 737 μm is reduced. The same considerations hold in the case that population of fines and fibres are considered.

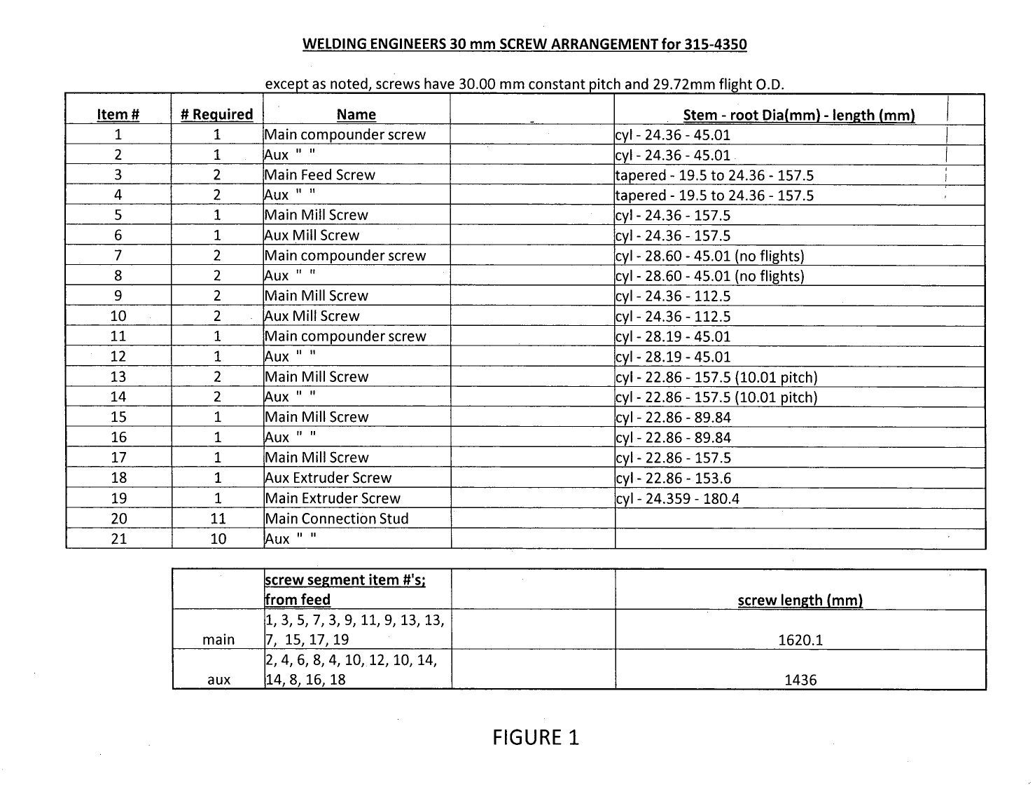

The plots and graphs also show the measurements of the thermally treated ligno-cellulosic biomass after fiber shives reduction, which in this case was passing it through a twin screw extruder at about 35% dry matter content having the screw element design of FIG. 1. The twin screw extruder is also known as a mechanical treatment or the application of mechanical forces on the thermally treated ligno-cellulosic biomass. One of ordinary skill could easily obtain this design from the manufacturer listed.

The dominant role of the fiber shives is evidenced by seeing that first, according to FIG. 4, the thermally treated ligno-cellulosic biomass after fiber shives reduction through the extruder has a reduced percent area of long fibres for both the low and high severity factors of 3.1 and 3.91. However, for the low severity factor of 3.1, the conversion of fiber shives improved the glucan accessibility from 84 to 93 percent (FIG. 2). Again, the same considerations hold in the case that population of fibres and fiber shives are considered. While at the high severity of 3.91, there was substantially no improvement in the glucan accessibility. Were the long fibres responsible for accessibility, the accessibility of the glucans for the thermally treated ligno-cellulosic biomass should have been less than 94% and the reduction of the percent area of long fibres (or equivalently the population of long fibres) during the extrusion (application of mechanical forces) should have caused an increase in the accessibility. The accessibility did not increase establishing that it is not the conversion of fibres to fines that causes the increased accessibility.

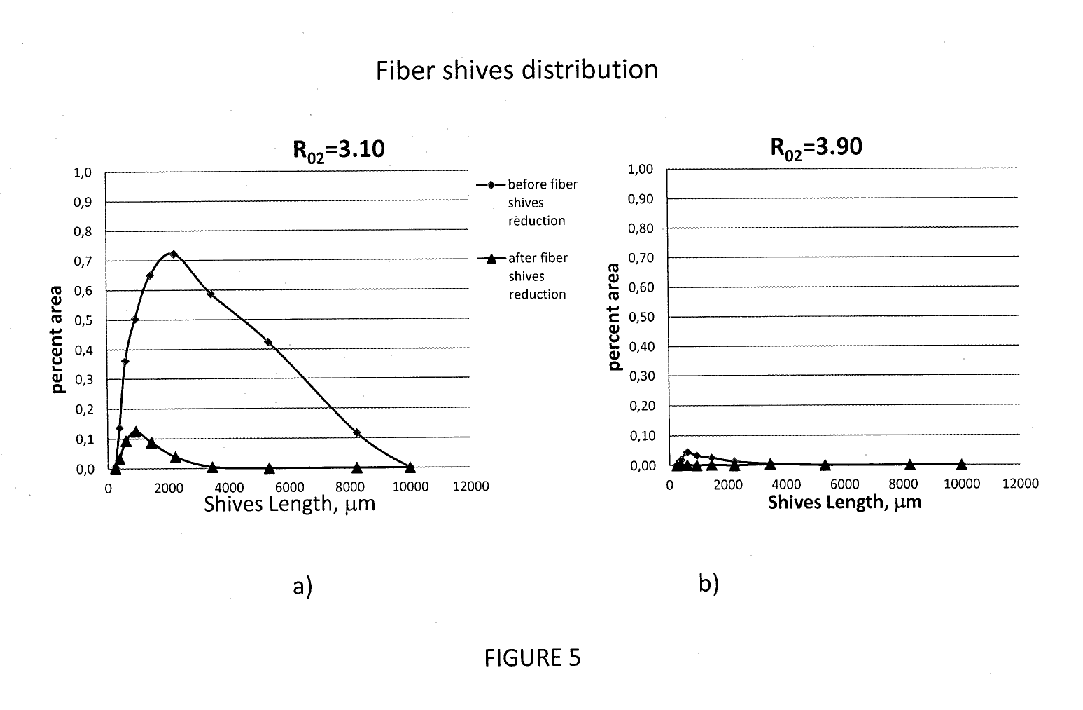

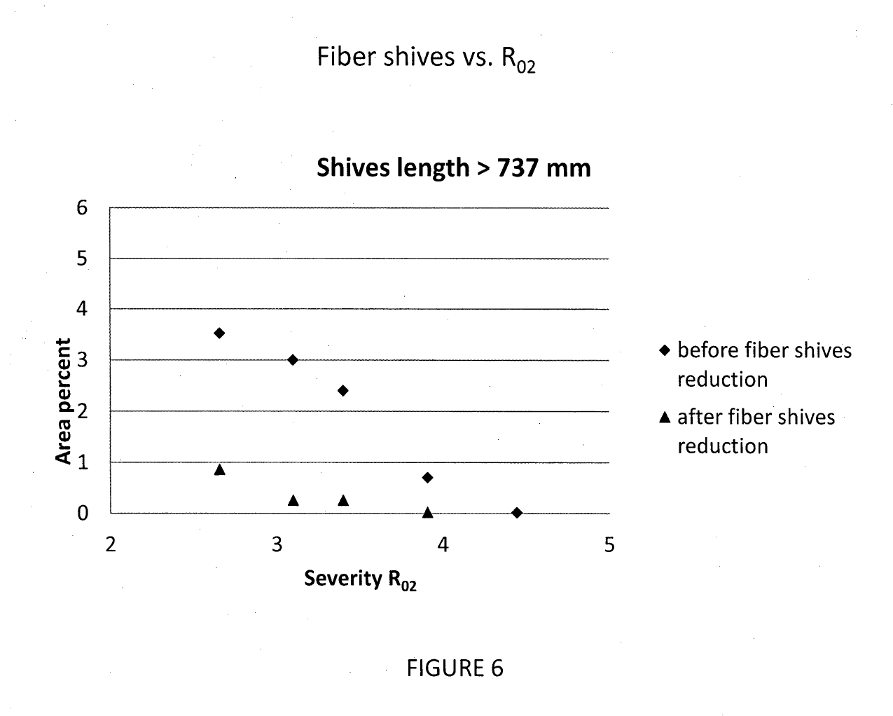

The role of the fiber shives is shown in FIG. 5, which contains the percent area distribution of fiber shives of two samples prepared at low severity factor (R₀₂=3.10, FIG. 5a) and high severity factor (R₀₂=3.90, FIG. 5b), before fiber shives reduction and after fiber shives reduction. The sample at low severity before fiber shives reduction contains a remarkable amount of fiber shives and the mechanical treatment reduces the amount of fiber shives in the sample at low severity, while the sample at high severity has already a small amount of fiber shives before fiber shives reduction. FIG. 6 reports the total percent area of fiber shives having a fiber shives length greater than 737 μm. The percent area of fiber shives of the sample at low severity is reduced from 3.5% to less than 1% by the fiber shives reduction. However, for the high severity thermally treated ligno-cellulosic biomass, fiber shives percent area is already less than 1% before fiber shives reduction. Thus, there is the conclusion that once the fiber shives are below a certain threshold, their removal does not impact the properties in a measurable way. Therefore, the percent area of the fiber shives having a shive length greater than or equal to 737 μm relative to the total area of fiber shives, fibres and fines of the thermally treated ligno-cellulosic biomass before fiber shives reduction is greater than a value selected from the group consisting of 1%, 2%, 3% and 4% and the percent area of the fiber shives having a shive length greater than or equal to 737 μm relative to the total area of fiber shives, fibers and fines of the thermally treated ligno-cellulosic biomass after fiber shives reduction is less than a value selected from the group consisting of 1%, 0.5, 0.25%, 0.02%, and 0.1%.

In a preferred embodiment, the percent area of the fiber shives having a shive length greater than or equal to 737 μm relative to the total area of fiber shives, fibers and fines of the thermally treated ligno-cellulosic biomass after fiber shives reduction is greater than 0, and less than a value selected from the group consisting of 1%, 0.5, 0.25%, 0.02%, and 0.1%, that is some long fiber shives are still present in the thermally treated ligno-cellulosic biomass after fiber shives reduction.

The total area of fiber shives, fibres and fines is measured using automated optical analysis which determines the area of the fiber shives, the area of the fibres and the area of fines. The proper machine, as described in the experimental section, will often provide the area of each individual class, as well as the area of each class as a percent of the total area of the sum of the classes. In the event the machine does not do the math, one of ordinary skill should be able to calculate the percent area knowing the areas, or the area knowing the total area and percent of each class measured.

In any event, the effect of the shives reduction should be such that the percent area of the fiber shives having a shive length greater than or equal to 737 μm relative to the total area of fiber shives, fibres and fines of the thermally treated ligno-cellulosic biomass after fiber shives reduction is less than a value selected from the group consisting of 5%, 10%, 20%, 30%, 40%, 50%, 60% and 70% of the percent area of the fiber shives having a shive length greater than or equal to 737 μm relative to the total area of fiber shives, fibres and fines of the thermally treated ligno-cellulosic biomass before fiber shives reduction.

Because the fiber shives are comprised of fibre bundles and agglomerated fibres, a reduced amount of energy is needed as compared to the prior art. As described in the experimental section only 0.1 to 0.2 Kw-h/kg on a wet basis or 0.25 to 0.50 Kw-h/kg on a dry matter basis was used to achieve the effects. Thus the preferred amount of work, or energy, imparted to the thermally treated ligno-cellulosic biomass is preferably less than a number selected from the group consisting of 500 Wh/Kg, 400 Wh/Kg, 300 Wh/Kg, 200 Wh/Kg, 100 Wh/Kg, per kg of the thermally treated ligno-cellulosic biomass on a dry basis. It is preferable that at least a part of the fiber shives reduction is done by applying mechanical forces to the thermally treated ligno-cellulosic biomass, and all the work applied in form of mechanical forces on the thermally treated ligno-cellulosic biomass is less than 500 Wh/Kg per kg of the thermally treated ligno-cellulosic biomass on a dry basis. It is even more preferable that all the work done by all the forms of mechanical forces on the thermally treated ligno-cellulosic biomass is less than a value selected from the group consisting of 400 Wh/Kg, 300 Wh/Kg, 200 Wh/Kg, 100 Wh/Kg, per kg of the thermally treated ligno-cellulosic biomass on a dry basis.

The application of mechanical forces to the thermally treated ligno-cellulosic biomass should be a mechanical process or sub-processes which applies work to the thermally treated ligno-cellulosic biomass and reduces the number of fiber shives longer than or equal to 737 μm during the fiber shives reduction. Mechanical forces applying work are distinct from chemical processes which may dissolve the fiber shives, for example. The type of forces or work applied as a mechanical force is shear, compression, and moving. It should be appreciated that the mechanical treatment may be a conversion process where the application of mechanical forces converts at least a portion of the fiber shives in the thermally treated ligno-cellulosic biomass to fibres or fines that remain part of the output. One class of machines for applying this type of work in a mechanical manner are those machines which apply shear such as an extruder, a twin screw extruder, a co-rotating extruder, a counter-rotating twin screw extruder, a disk mill, a bunbury, a grinder, a rolling mill, a hammer mill.

Preferably, the mechanical energy applied to the thermally treated ligno-cellulosic biomass is not mechanical energy derived from free-fall or gravity mixing.

In any case, it is noted the amount of work applied to the thermally treated ligno-cellulosic biomass for a given amount of time should be greater than the amount of work that can be provided by the forces of gravity or free fall mixing in that same period. One way to measure this is to consider the period of time in which the fiber shives are reduced to be the called fiber shives reduction time. The amount of work applied to the thermally treated ligno-cellulosic biomass during the fiber shives reduction time is preferably greater than the amount of work which can be applied to the thermally treated ligno-cellulosic biomass by free fall mixing or gravity. One embodiment will have no work applied in the form of free fall mixing or gravity during the shives reduction.

The fiber shives reduction time is preferably in the range of 0.1 to 30 minutes. While the fiber shives reduction time can be any positive amount less than 12 hours, less than 6 hours is more preferable, with less than 3 hours even more preferred and less than 1 hour more preferred, and less than 30 minutes being more preferable with less than 20 minutes being most preferred. In the case of an extruder, the preferred fiber shives reduction time is in the range of 0.1 to 15 minutes.

One of ordinary skill knowing that the forces are to be applied to fibre shives which on the average are 2 to 5 times the width of the fibre (less than or equal to 75 μm, averaging of 30-40 μm versus the fiber shives of 130-180 μm width) can easily adjust the apparatus. The twin screw extruder applies mechanical work in the forms of shear, compression and movement down the barrel of the screw. For a twin screw extruder one keeps the flights and distances further apart, as tighter distances applying forces to fibres are only wasted. In the experiments conducted in this specification, a conventional twin screw extruder for PET resins was used with no special screw as described in the prior art. For mills or blades, one sets the distance between the two parts creating the force for the particles having width of 130-180 μm, not the particles less than or equal to 75 μm.

The simplest example of these machines are grist mills where two stones are rotated with a space between them. The space between the stones sets the size. One of ordinary skill would set the stones a distance apart to apply the force to particles having a width of >75 μm, with the fibres having a width of less than 75 μm passing between the stones with little or no work applied to these smaller particles. A disk mill is of the similar operation as it is the space between the disks which sets the application of the force.

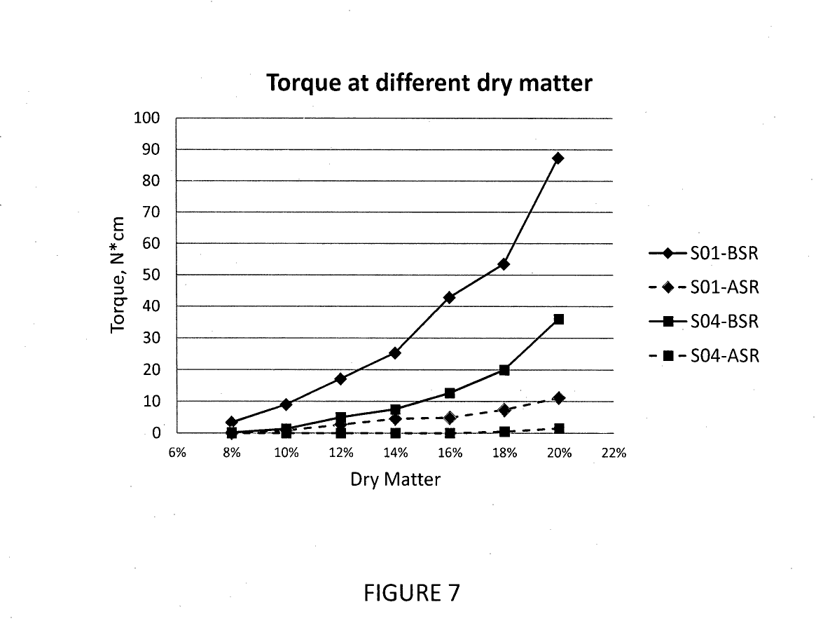

An additional feature it has been discovered, that once the fiber shives level is low enough, the thermally treated ligno-cellulosic biomass after fiber shives reduction will have much lower viscosity than the thermally treated ligno-cellulosic biomass when both are made into a slurry of water at the same dry matter content. FIG. 7 demonstrates this, at 20% dry matter the S01 (produced at a steam explosion severity factor of 2.66)) thermally treated material before fiber shives reduction needed a torque of 87 N-cm, while the thermally treated ligno-cellulosic biomass after shives reduction, needed only 11 N-cm.

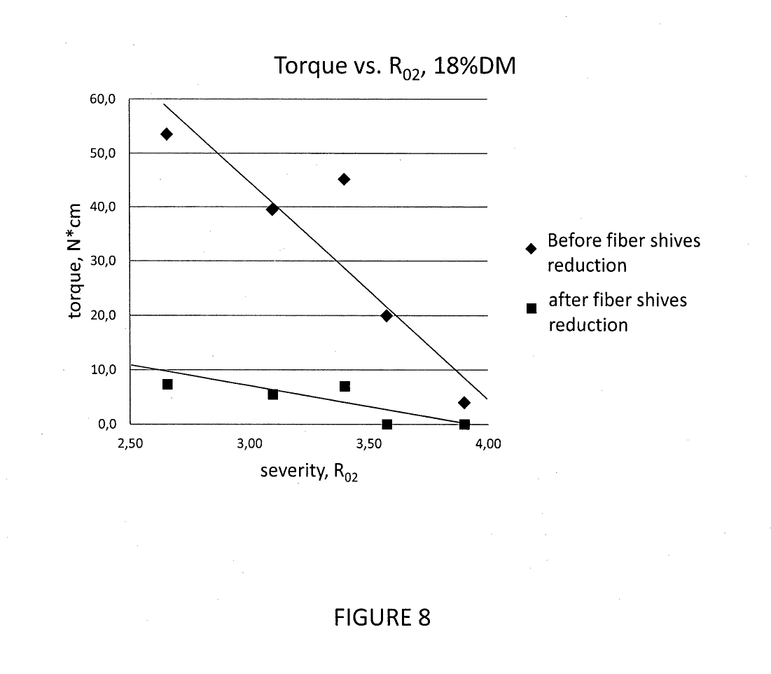

FIG. 8 shows the torque needed to agitate a slurry at 18% dry matter of thermally treated materials prepared at different severity factor, before and after fiber shives reduction. The torque decreases by increasing the severity factor, as the samples at low severity factor contain a bigger amount of fiber shives (FIG. 6). For each thermally treated material, the torque decreases by reducing the fiber shives by means of a mechanical treatment, but the effect is remarkably more evident in samples at low severity factor, which contains more fiber shives.

This slurry effect is especially critical as it can be can be done without hydrolysis, meaning that the low viscosity stream can be passed over an immobilized enzyme bed for enzymatic hydrolysis, or passed over a ion exchange resin for cationic exchange and subsequent “acid” hydrolysis.

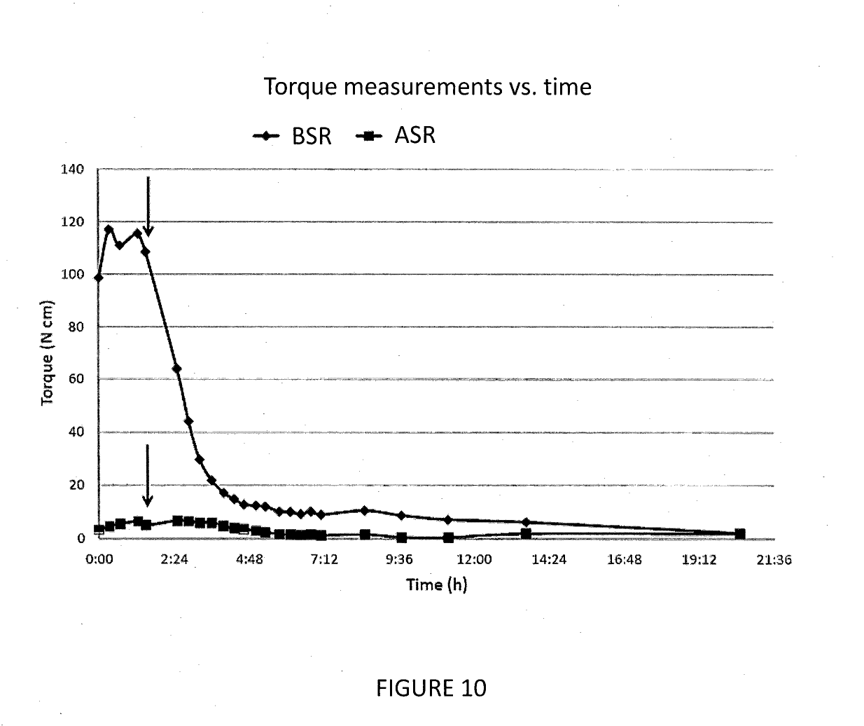

This property is especially useful when exposing the material to enzymatic hydrolysis. In FIG. 10, the thermally treated ligno-cellulosic biomass before fiber shives reduction and the thermally treated ligno-cellulosic biomass after shives reduction were “slurried” into water with enzymes added at the arrow. It took 2+ hours after the enzymes were added for the viscosity of the thermally treated ligno-cellulosic biomass before fiber shives reduction to approach that of the thermally treated ligno-cellulosic biomass after fiber shives reduction. Thus, the process can be further characterized in that the output of thermally treated ligno-cellulosic biomass after fiber shives reduction is characterized by having a viscosity of a slurry of the thermally treated ligno-cellulosic biomass after fiber shives reduction in water less than the viscosity of a slurry of the thermally treated ligno-cellulosic biomass before fiber shives reduction in water, wherein the viscosities are measured at 25° C., at a shear rate of 10 s⁻¹ and at a dry matter content of 7% by weight of each slurry.

The process can be further characterized in that the thermally treated ligno-cellulosic biomass after fiber shives reduction is characterized by having a viscosity of a slurry of the thermally treated ligno-cellulosic biomass after fiber shives reduction in water less than a value selected from the group consisting of 0.1 Pa s, 0.3 Pa s, 0.5 Pa s, 0.7 Pa s, 0.9 Pa s, 1.0 Pa s, 1.5 Pa s, 2.0 Pa s, 2.5 Pa s, 3.0 Pa s, 4 Pa s, 5 Pa s, 7 Pa s, 9 Pa s, 10 Pa s, wherein the viscosity is measured at 25° C., at a shear rate of 10 s⁻¹ and at a dry matter content of 7% by weight of the slurry of the thermally treated ligno-cellulosic biomass after fiber shives reduction in the water.

The process can further comprise a slurry step, wherein the thermally treated ligno-cellulosic biomass before, during or after fiber shives reduction is dispersed into a liquid carrier, preferably comprising water or aqueous, to create a slurry stream. The slurry stream preferably has a viscosity less than a value selected from the group consisting of 0.1 Pa s, 0.3 Pa s, 0.5 Pa s, 0.7 Pa s, 0.9 Pa s, 1.0 Pa s, 1.5 Pa s, 2.0 Pa s, 2.5 Pa s, 3.0 Pa s, 4 Pa s, 5 Pa s, 7 Pa s, 9 Pa s, 10 Pa s, wherein the viscosity is measured at 25° C., at a shear rate of 10 s⁻¹ and at a dry matter content of 7% by weight of the slurry stream. The slurry stream will preferably have a dry matter content less than 100% but greater than a value selected from the group consisting of 5%, 7%, 8%, 10%, 12%, 15%, 18%, 20%, 25%, 30%, 35%, and 40%.

Because this slurry stream having this viscosity can be made without the use of hydrolysis catalysts such as enzymes, acids or bases, thus, the inventors have discovered an entirely new article of manufacture which is a slurry comprising water, soluble sugars, solid lignin, solid cellulose, which has a dry matter content in the range of 20 to 80% by weight of the total amount of the slurry and is void of or substantially void of a hydrolytic catalyst such as an enzyme or enzymes. Other preferable ranges of dry matter range are 25 to 80% by weight, with 30 to 80% by weight even more preferable. In some instances the dry matter range will have an upper limit of 70% by weight, with 60% less preferable and 40% even less preferable.

The torque of the slurry comprising the thermally ligno-cellulosic biomass after fiber shives reduction at 10 minutes after the addition of the solvent is less than the torque of a mixture of the thermally treated ligno-cellulosic biomass before fiber shives reduction when using the same amount and composition of the solvent measured 10 minutes after the solvent has been added to the thermally pre-treated ligno-cellulosic biomass before fiber shives reduction and under the same mixing condition when both torque measurements are at 25° C. Preferably the torque of the thermally treated ligno-cellulosic biomass after fiber shives reduction should be at least less than 50% of the torque of the thermally treated ligno-cellulosic biomass before fiber shives reduction, with at least less than 40% even more preferred, with at least less than 30% even more preferred.

It is also preferable that the solvent creating the slurry is not pure recycled process water as offered in WO 2011/044292 and WO 2011/044282, but to use liquid containing solubles and possibly insolubles from a hydrolysis reactor or alternatively use materials derived from the stillage after the hydrolyzed material has been fermented. In another embodiment, the solvent comprises liquids produced during the thermal treatment, said liquids comprising monomeric and oligomeric sugars which have been solubilized as an effect of the thermal treatment. While the addition point in WO 2011/044292 and WO 2011/044282 is at the end of a compounder, the liquid comprising the hydrolysis products of a similarly, if not same, ligno-cellulosic biomass, also considered a solvent in this specification is used to slurry the thermally treated ligno-cellulosic biomass after fiber shives reduction.

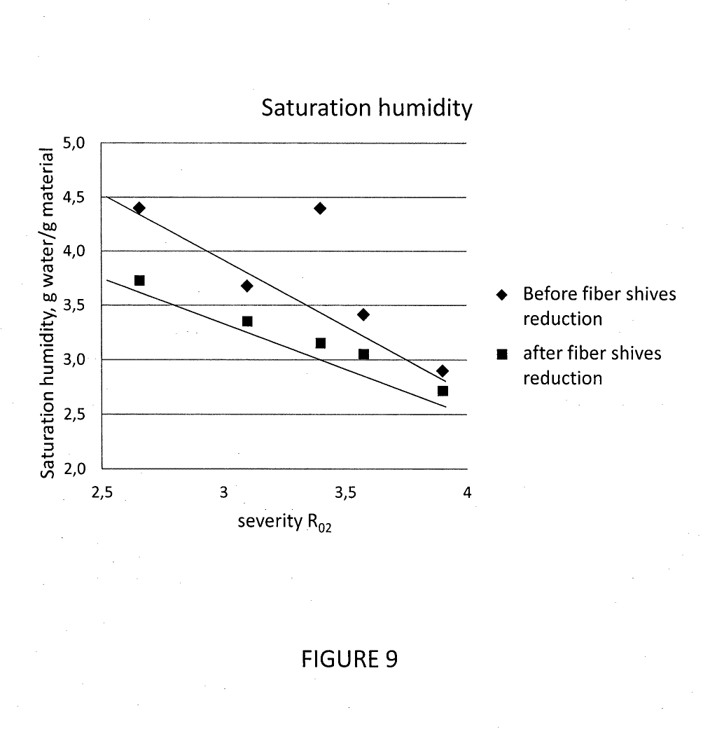

The process can be further characterized, as demonstrated in FIG. 9, by the saturation humidity of the thermally treated ligno-cellulosic biomass after fiber shives reduction and the thermally treated ligno-cellulosic biomass before fiber shives reduction because the saturation humidity of the thermally treated ligno-cellulosic biomass after fiber shives reduction is less than the saturation humidity of thermally treated ligno-cellulosic biomass.

It can be said that thermally treated ligno-cellulosic biomass after fiber shives reduction has a first saturation humidity, and the thermally treated ligno-cellulosic biomass before fiber shives reduction has a second saturation humidity, and the first saturation humidity is less than the second saturation humidity.

In fact, when compared to each other the saturation humidity of the thermally treated ligno-cellulosic biomass after fiber shives reduction is less than a value selected from the group consisting of 20%, 30%, 40%, 50%, 60%, 70% and 80% of the thermally treated ligno-cellulosic biomass before fiber shives reduction.

In terms of output characterization, the saturation humidity of the thermally treated ligno-cellulosic biomass after fiber shives reduction is preferably less than a value selected from the group consisting of 5.5, 5.0, 4.5, 4.0, 3.5, 3.0, 2.5, 2.0, 1.5, and 1.0 g/g expressed as gram of water per gram of thermally treated ligno-cellulosic biomass after fiber shives reduction on a dry basis.

In terms of feedstock selection it is preferable that the saturation humidity of the thermally treated ligno-cellulosic biomass before fiber shives reduction is less than a value selected from the group consisting of 6.0, 5.5, 5.0, 4.5, 4.0, 3.5, 3.0, and 2.5 g/g, expressed as gram of water per gram of thermally treated ligno-cellulosic biomass ligno-cellulosic biomass on a dry basis.

The thermally treated ligno-cellulosic biomass preferably has a dry matter content of at least 20% by weight of the total content of the thermally treated ligno-cellulosic biomass. With the dry matter content of the thermally treated ligno-cellulosic biomass preferably in the range of at least a value selected from the group consisting of 25%, 30%, 35%, and 40% by weight of the total content of the thermally treated ligno-cellulosic biomass to less than 80% by weight of the total content of the thermally treated ligno-cellulosic biomass.

Xylose recovery is the percent ratio between the total amount of xylans in the thermally treated ligno-cellulosic biomass before fiber shives reduction (as xylose equivalents calculated including insoluble xylans, xylo-oligomers, xilobiose and xylose present in both the solid and liquid of the ligno-cellulosic biomass) and the total amount of xylans (converted in xylose equivalents) present in the raw material before the thermal treatment. The complementary to 100% of the xylose recovery represents therefore the total amount of xylans degradation products as an effect of the thermal treatment.

In the case when the fiber shives reduction converts fiber shives to fines or fibres, the amount of xylose equivalents in the final composition after fiber shives reduction is the same as the amount of xylose equivalents in the thermally treated material before fiber shives reduction.

In terms of xylose recovery, the thermally treated ligno-cellulosic biomass before fiber shives reduction may preferably have a xylose recovery greater than a value selected from the group consisting of 85%, 90%, 92%, 95%, and 98%.

Glucose recovery is the percent ratio between the total amount of glucans in the thermally treated ligno-cellulosic biomass before fiber shives reduction (as glucose equivalents calculated including insoluble glucans, gluco-oligomers, cellobiose and glucose present in both the solid and liquid of the ligno-cellulosic biomass) and the total amount of glucans (converted in glucose equivalents) present in the raw material before the thermal treatment. The complementary to 100% of the glucose recovery represents therefore the total amount of glucans degradation products as an effect of the thermal treatment.

In terms of glucose recovery, the thermally treated ligno-cellulosic biomass before fiber shives reduction preferably has a glucose recovery greater than a value selected from the group consisting of 90%, 92%, 95%, and 98%. The glucans accessibility of the thermally treated ligno-cellulosic biomass before fiber shives reduction is preferably greater than a value selected from the group consisting of 80%, 85%, 88%, 90%, 92%, 95%, and 98% or the glucans accessibility can be lower than a value selected from the group consisting of 75%, 78%, 80%, 82%, 85%, 88% and 91%.

Like xylose, in the case when the fiber shives reduction converts fiber shives to fines or fibres, the amount of glucose equivalents in the final composition after fiber shives reduction is the same as the amount of glucose equivalents in the thermally treated material before fiber shives reduction.

In terms of glucans accessibility, the thermally treated ligno-cellulosic biomass after fiber shives reduction has a first glucans accessibility and the thermally treated ligno-cellulosic biomass before fiber shives reduction has a second glucans accessibility and the first glucans accessibility is greater than the second glucans accessibility.

As the experiments in this specification were done without the addition of acids or bases, it can be said that the thermally treated ligno-cellulosic biomass may preferably be free of added ionic species such as acids or bases, which are species added to the thermally treated ligno-cellulosic biomass after harvesting, i.e. not part of its natural composition. Thus the thermally treated ligno-cellulosic biomass is free of an added acid and/or added base. It is preferred then that if there any ionic groups that the amount and type of ionic groups present in the ligno-cellulosic feedstock are the amounts and types of the respective ionic groups that are not derived from the group consisting of mineral acids, organic acids and organic bases.

The same is true of the process itself of thermal treatment and mechanical treatment as these steps can be conducted in the absence of an added acid and/or added base.

In particular, preferably the thermally treated ligno-cellulosic biomass does not contain sulfur. In the case that sulfur is already present in the ligno-cellulosic biomass feedstock, the percent amount of sulfur by weight in the thermally pretreated ligno-cellulosic biomass on a dry basis is preferably less than a value selected from the group consisting of 4%, 3%, 2, and 1%.

The thermal treatment preferably have a severity (R_{O}) lower than a value selected from the group consisting of 4.0, 3.75, 3.5, 3.25, 3.0, 2.75 and 2.5. The preferred thermal treatment will also comprise a steam explosion step.

In a preferred embodiment, the thermal treatment is conducted at low severity factor, so as to enhance the fiber shives reduction effects in the thermally treated ligno-cellulosic material after fiber shives reduction with respect to the thermally treated ligno-cellulosic biomass before fiber shives reduction. Moreover, the low severity thermal treatment will be more convenient, as it requires less thermal energy. As a consequence the low severity thermally treated ligno-cellulosic biomass after fiber shives reduction will have some peculiar properties.

It is known in the art that a severe thermal treatment has a more remarkable effect on xylans, in terms of solubilization and/or degradation, than on glucans. Thereby, the low severity thermally treated ligno-cellulosic biomass will contain more xylans, with respect to glucans, than a high severity thermally treated ligno-cellulosic biomass, as evident in FIG. 3. This is evident in the graph of FIG. 3. The fiber shives reduction step is conducted substantially to not change the chemical composition of the thermally treated ligno-cellulosic biomass, thereby the thermally treated ligno-cellulosic biomass, either before and after fiber shives reduction, may be characterized by having a percent ratio of the amount of xylans to the amount of glucans which is greater than 5%, more preferably greater than 10%, even more preferably greater than 15%, even more preferably greater than 20%, even yet more preferably greater than 25%, and most preferably greater than 30%. On the other hand, less xylans and glucans degradation product, such as furfural and HMF, will be generated in the thermal treatment.

### Low Viscosity Slurry

The formation of a slurry requires the dispersion of the thermally treated ligno-cellulosic biomass in a liquid carrier, wherein the dispersion may occur before, during or after the fiber shives reduction step.

In an embodiment, the carrier liquid is added to the thermally treated ligno-cellulosic biomass after fiber shives reduction.

In another embodiment, is the thermally treated ligno-cellulosic biomass after fiber shives reduction to be added to the carrier liquid.

In another embodiment, is the thermally treated ligno-cellulosic biomass before or during fiber shives reduction to be added to the carrier liquid, and then subjected to fiber shives reduction, for instance by means of a disk refiner or an apparatus to remove shives.

In yet another embodiment, the carrier liquid is added to the thermally treated ligno-cellulosic biomass before or during fiber shives reduction.

Mixing may be applied to promote the dispersion of the treated biomass in the liquid carrier.

In preferred embodiment, the treated biomass is inserted in a vessel and a carrier liquid comprised of water is added to reach a desired dry matter content by weight in the mixture. Liquid may be added, partly or in its entirety, before the insertion into the vessel. Added liquid may be added before or during mixing. Added liquid is preferably added in a continuous way. In one embodiment, the final dry matter in the mixture is 15% or greater and described in further detail below.

In one embodiment, the added liquid carrier comprises water. The added liquid carrier may comprise liquids produced from the thermal treatment of the ligno-cellulosic biomass feedstock, wherein said liquids eventually comprises also undissolved particles of the feedstock. In a preferred embodiment, the added carrier liquid may also comprise dissolved sugars from the thermally treated biomass before or after fiber shives reduction. In another embodiment, the carrier liquid may also comprise soluble species obtainable from either a previously liquefied slurry of the treated ligno-cellulosic biomass after fiber shives reduction or the hydrolysis of the treated ligno-cellulosic biomass after fiber shives reduction. The carrier liquid may or may not contain a hydrolysis catalyst such as an enzyme which hydrolyses the cellulose into glucose In various embodiment, additives may be present in the carrier liquid. Preferably, low shear mixing condition are applied to the mixture, for instance by means of a Rushton impeller. A person skilled in the art knows how to properly apply a low shear to a mixture, by selecting setup and mixing parameters.

As stated previously, the inventors surprisingly discovered that once the carrier liquid contacts the thermally treated ligno-cellulosic biomass after fiber shives reduction, the dispersion of the thermally treated ligno-cellulosic biomass into the carrier liquid proceeds quickly. This is immediately seen by comparing the torque applied to a stirrer disposed in the produced slurry, described as the applied torque, with the applied torque of thermally ligno-cellulosic biomass which has not been subjected to fiber shives reduction, which has also been combined with the carrier liquid, at the same dry weight percent.

### Feedstock Selection

Because the feedstock may use naturally occurring ligno-cellulosic biomass, the stream will have relatively young carbon materials. The following, taken from ASTM D 6866-04 describes the contemporary carbon, which is that found in bio-based hydrocarbons, as opposed to hydrocarbons derived from oil wells, which was derived from biomass thousands of years ago. “[A] direct indication of the relative contribution of fossil carbon and living biospheric carbon can be as expressed as the fraction (or percentage) of contemporary carbon, symbol f_{C}. This is derived from f_{M} through the use of the observed input function for atmospheric ¹⁴C over recent decades, representing the combined effects of fossil dilution of the ¹⁴C (minor) and nuclear testing enhancement (major). The relation between f_{C} and f_{M} is necessarily a function of time. By 1985, when the particulate sampling discussed in the cited reference [of ASTM D 6866-04, the teachings of which are incorporated by reference in their entirety] the f_{M} ratio had decreased to ca. 1.2.”

Fossil carbon is carbon that contains essentially no radiocarbon because its age is very much greater than the 5730 year half life of ¹⁴C. Modern carbon is explicitly 0.95 times the specific activity of SRM 4990b (the original oxalic acid radiocarbon standard), normalized to δ¹³C=−19%. Functionally, the faction of modern carbon=(1/0.95) where the unit 1 is defined as the concentration of ¹⁴C contemporaneous with 1950 [A.D.] wood (that is, pre-atmospheric nuclear testing) and 0.95 are used to correct for the post 1950 [A.D.] bomb ¹⁴C injection into the atmosphere. As described in the analysis and interpretation section of the test method, a 100% ¹⁴C indicates an entirely modern carbon source, such as the products derived from this process. Therefore, the percent ¹⁴C of the product stream from the process will be at least 75%, with 85% more preferred, 95% even preferred and at least 99% even more preferred and at least 100% the most preferred. (The test method notes that the percent ¹⁴C can be slightly greater than 100% for the reasons set forth in the method). These percentages can also be equated to the amount of contemporary carbon as well.

Therefore the amount of contemporary carbon relative to the total amount of carbon is preferred to be at least 75%, with 85% more preferred, 95% even more preferred and at least 99% even more preferred and at least 100% the most preferred. Correspondingly, each carbon containing compound in the reactor, which includes a plurality of carbon containing conversion products will have an amount of contemporary carbon relative to total amount of carbon is preferred to be at least 75%, with 85% more preferred, 95% even preferred and at least 99% even more preferred and at least 100% the most preferred.

In general, a natural or naturally occurring ligno-cellulosic biomass can be one feed stock for this process. Ligno-cellulosic materials can be described as follows:

Apart from starch, the three major constituents in plant biomass are cellulose, hemicellulose and lignin, which are commonly referred to by the generic term lignocellulose. Polysaccharide-containing biomasses as a generic term include both starch and ligno-cellulosic biomasses. Therefore, some types of feedstocks can be plant biomass, polysaccharide containing biomass, and ligno-cellulosic biomass.

Polysaccharide-containing biomasses according to the present invention include any material containing polymeric sugars e.g. in the form of starch as well as refined starch, cellulose and hemicellulose.

Relevant types of naturally occurring biomasses for deriving the claimed invention may include biomasses derived from agricultural crops selected from the group consisting of starch containing grains, refined starch; corn stover, bagasse, straw e.g. from rice, wheat, rye, oat, barley, rape, sorghum; softwood e.g. Pinus sylvestris, Pinus radiate; hardwood e.g. Salix spp. Eucalyptus spp.; tubers e.g. beet, potato; cereals from e.g. rice, wheat, rye, oat, barley, rape, sorghum and corn; waste paper, fiber fractions from biogas processing, manure, residues from oil palm processing, municipal solid waste or the like. Although the experiments are limited to a few examples of the enumerated list above, the invention is believed applicable to all because the characterization is primarily to the unique characteristics of the lignin and surface area.

The ligno-cellulosic biomass feedstock used to derive the composition is preferably from the family usually called grasses. The proper name is the family known as Poaceae or Gramineae in the Class Liliopsida (the monocots) of the flowering plants. Plants of this family are usually called grasses, or, to distinguish them from other graminoids, true grasses. Bamboo is also included. There are about 600 genera and some 9,000-10,000 or more species of grasses (Kew Index of World Grass Species).

Poaceae includes the staple food grains and cereal crops grown around the world, lawn and forage grasses, and bamboo. Poaceae generally have hollow stems called culms, which are plugged (solid) at intervals called nodes, the points along the culm at which leaves arise. Grass leaves are usually alternate, distichous (in one plane) or rarely spiral, and parallel-veined. Each leaf is differentiated into a lower sheath which hugs the stem for a distance and a blade with margins usually entire. The leaf blades of many grasses are hardened with silica phytoliths, which helps discourage grazing animals. In some grasses (such as sword grass) this makes the edges of the grass blades sharp enough to cut human skin. A membranous appendage or fringe of hairs, called the ligule, lies at the junction between sheath and blade, preventing water or insects from penetrating into the sheath. Grass blades grow at the base of the blade and not from elongated stem tips. This low growth point evolved in response to grazing animals and allows grasses to be grazed or mown regularly without severe damage to the plant.

Flowers of Poaceae are characteristically arranged in spikelets, each spikelet having one or more florets (the spikelets are further grouped into panicles or spikes). A spikelet consists of two (or sometimes fewer) bracts at the base, called glumes, followed by one or more florets. A floret consists of the flower surrounded by two bracts called the lemma (the external one) and the palea (the internal). The flowers are usually hermaphroditic (maize, monoecious, is an exception) and pollination is almost always anemophilous. The perianth is reduced to two scales, called lodicules, that expand and contract to spread the lemma and palea; these are generally interpreted to be modified sepals.

The fruit of Poaceae is a caryopsis in which the seed coat is fused to the fruit wall and thus, not separable from it (as in a maize kernel).

There are three general classifications of growth habit present in grasses; bunch-type (also called caespitose), stoloniferous and rhizomatous.

The success of the grasses lies in part in their morphology and growth processes, and in part in their physiological diversity. Most of the grasses divide into two physiological groups, using the C3 and C4 photosynthetic pathways for carbon fixation. The C4 grasses have a photosynthetic pathway linked to specialized Kranz leaf anatomy that particularly adapts them to hot climates and an atmosphere low in carbon dioxide.

C3 grasses are referred to as “cool season grasses” while C4 plants are considered “warm season grasses”. Grasses may be either annual or perennial. Examples of annual cool season are wheat, rye, annual bluegrass (annual meadowgrass, Poa annua and oat). Examples of perennial cool season are orchard grass (cocksfoot, Dactylis glomerata), fescue (Festuca spp), Kentucky Bluegrass and perennial ryegrass (Lolium perenne). Examples of annual warm season are corn, sudangrass and pearl millet. Examples of Perennial Warm Season are big bluestem, indian grass, bermuda grass and switch grass.

One classification of the grass family recognizes twelve subfamilies: These are 1) anomochlooideae, a small lineage of broad-leaved grasses that includes two genera (Anomochloa, Streptochaeta); 2) Pharoideae, a small lineage of grasses that includes three genera, including Pharus and Leptaspis; 3) Puelioideae a small lineage that includes the African genus Puelia; 4) Pooideae which includes wheat, barley, oats, brome-grass (Bronnus) and reed-grasses (Calamagrostis); 5) Bambusoideae which includes bamboo; 6) Ehrhartoideae, which includes rice, and wild rice; 7) Arundinoideae, which includes the giant reed and common reed; 8) Centothecoideae, a small subfamily of 11 genera that is sometimes included in Panicoideae; 9) Chloridoideae including the lovegrasses (Eragrostis, ca. 350 species, including teff), dropseeds (Sporobolus, some 160 species), finger millet (Eleusine coracana (L.) Gaertn.), and the muhly grasses (Muhlenbergia, ca. 175 species); 10) Panicoideae including panic grass, maize, sorghum, sugar cane, most millets, fonio and bluestem grasses; 11) Micrairoideae and 12) Danthoniodieae including pampas grass; with Poa which is a genus of about 500 species of grasses, native to the temperate regions of both hemispheres.

Agricultural grasses grown for their edible seeds are called cereals. Three common cereals are rice, wheat and maize (corn). Of all crops, 70% are grasses.

Sugarcane is the major source of sugar production. Grasses are used for construction. Scaffolding made from bamboo is able to withstand typhoon force winds that would break steel scaffolding.

Larger bamboos and Arundo donax have stout culms that can be used in a manner similar to timber, and grass roots stabilize the sod of sod houses. Arundo is used to make reeds for woodwind instruments, and bamboo is used for innumerable implements.

Another naturally occurring ligno-cellulosic biomass feedstock may be woody plants or woods. A woody plant is a plant that uses wood as its structural tissue. These are typically perennial plants whose stems and larger roots are reinforced with wood produced adjacent to the vascular tissues. The main stem, larger branches, and roots of these plants are usually covered by a layer of thickened bark. Woody plants are usually either trees, shrubs, or lianas. Wood is a structural cellular adaptation that allows woody plants to grow from above ground stems year after year, thus making some woody plants the largest and tallest plants.

These plants need a vascular system to move water and nutrients from the roots to the leaves (xylem) and to move sugars from the leaves to the rest of the plant (phloem). There are two kinds of xylem: primary that is formed during primary growth from procambium and secondary xylem that is formed during secondary growth from vascular cambium.

What is usually called “wood” is the secondary xylem of such plants.

The two main groups in which secondary xylem can be found are:
1) conifers (Coniferae): there are some six hundred species of conifers. All species have secondary xylem, which is relatively uniform in structure throughout this group. Many conifers become tall trees: the secondary xylem of such trees is marketed as softwood.
2) angiosperms (Angiospermae): there are some quarter of a million to four hundred thousand species of angiosperms. Within this group secondary xylem has not been found in the monocots (e.g. Poaceae). Many non-monocot angiosperms become trees, and the secondary xylem of these is marketed as hardwood.

The term softwood useful in this process is used to describe wood from trees that belong to gymnosperms. The gymnosperms are plants with naked seeds not enclosed in an ovary. These seed “fruits” are considered more primitive than hardwoods. Softwood trees are usually evergreen, bear cones, and have needles or scale like leaves. They include conifer species e.g. pine, spruces, firs, and cedars. Wood hardness varies among the conifer species.

The term hardwood useful for this process is used to describe wood from trees that belong to the angiosperm family. Angiosperms are plants with ovules enclosed for protection in an ovary. When fertilized, these ovules develop into seeds. The hardwood trees are usually broad-leaved; in temperate and boreal latitudes they are mostly deciduous, but in tropics and subtropics mostly evergreen. These leaves can be either simple (single blades) or they can be compound with leaflets attached to a leaf stem. Although variable in shape all hardwood leaves have a distinct network of fine veins. The hardwood plants include e.g. Aspen, Birch, Cherry, Maple, Oak and Teak. Therefore a preferred naturally occurring ligno-cellulosic biomass may be selected from the group consisting of the grasses and woods. Another preferred naturally occurring ligno-cellulosic biomass can be selected from the group consisting of the plants belonging to the conifers, angiosperms, Poaceae and families. Another preferred naturally occurring ligno-cellulosic biomass may be that biomass having at least 10% by weight of it dry matter as cellulose, or more preferably at least 5% by weight of its dry matter as cellulose.

The carbohydrate(s) comprising the invention is selected from the group of carbohydrates based upon the glucose, xylose, and mannose monomers and mixtures thereof.

The feedstock comprising lignin can be naturally occurring ligno-cellulosic biomass that has been ground to small particles, or one which has been further processed. One process for creating the feedstock comprising lignin, comprises the following steps.

### Preferable Pretreatment

It has been theorized that pretreatment of the feedstock is a solution to the challenge of processing an insoluble solid feedstock comprising lignin or polysaccharides in a pressurized environment. According to US 2011/0312051, sizing, grinding, drying, hot catalytic treatment and combinations thereof are suitable pretreatment of the feedstock to facilitate the continuous transporting of the feedstock. While not presenting any experimental evidence, US 2011/0312051 claims that mild acid hydrolysis of polysaccharides, catalytic hydrogenation of polysaccharides, or enzymatic hydrolysis of polysaccharides are all suitable to create a transportable feedstock. US 2011/0312051 also claims that hot water treatment, steam treatment, thermal treatment, chemical treatment, biological treatment, or catalytic treatment may result in lower molecular weight polysaccharides and depolymerized lignins that are more easily transported as compared to the untreated ones. While this may help transport, there is no disclosure or solution to how to pressurize the solid/liquid slurry resulting from the pre-treatment. In fact, as the inventors have learned the conventional wisdom and conventional systems used for pressuring slurries failed when pre-treated ligno-cellulosic biomass feedstock is used.

In the integrated second generation industrial operations, pre-treatment is often used to ensure that the structure of the ligno-cellulosic content is rendered more accessible to the catalysts, such as enzymes, and at the same time the concentrations of harmful inhibitory by-products such as acetic acid, furfural and hydroxymethyl furfural remain substantially low. There are several strategies to achieve increased accessibility, many of which may yet be invented.

The current pre-treatment strategies imply subjecting the ligno-cellulosic biomass material to temperatures between 110-250° C. for 1-60 min e.g.:

Hot water extraction
Multistage dilute acid hydrolysis, which removes dissolved material before inhibitory substances are formed
Dilute acid hydrolyses at relatively low severity conditions
Alkaline wet oxidation
Steam explosion.

A preferred pretreatment of a naturally occurring ligno-cellulosic biomass includes a soaking of the naturally occurring ligno-cellulosic biomass feedstock and a steam explosion of at least a part of the soaked naturally occurring ligno-cellulosic biomass feedstock.

The soaking occurs in a substance such as water in either vapor form, steam, or liquid form or liquid and steam together, to produce a product. The product is a soaked biomass containing a first liquid, with the first liquid usually being water in its liquid or vapor form or some mixture.

This soaking can be done by any number of techniques that expose a substance to water, which could be steam or liquid or mixture of steam and water, or, more in general, to water at high temperature and high pressure. The temperature should be in one of the following ranges: 145 to 165° C., 120 to 210° C., 140 to 210° C., 150 to 200° C., 155 to 185° C., 160 to 180° C. Although the time could be lengthy, such as up to but less than 24 hours, or less than 16 hours, or less than 12 hours, or less than 9 hours, or less than 6 hours; the time of exposure is preferably quite short, ranging from 1 minute to 6 hours, from 1 minute to 4 hours, from 1 minute to 3 hours, from 1 minute to 2.5 hours, more preferably 5 minutes to 1.5 hours, 5 minutes to 1 hour, 15 minutes to 1 hour.

If steam is used, it is preferably saturated, but could be superheated. The soaking step can be batch or continuous, with or without stirring. A low temperature soak prior to the high temperature soak can be used. The temperature of the low temperature soak is in the range of 25 to 90° C. Although the time could be lengthy, such as up to but less than 24 hours, or less than 16 hours, or less than 12 hours, or less than 9 hours or less than 6 hours; the time of exposure is preferably quite short, ranging from 1 minute to 6 hours, from 1 minute to 4 hours, from 1 minute to 3 hours, from 1 minute to 2.5 hours, more preferably 5 minutes to 1.5 hours, 5 minutes to 1 hour, 15 minutes to 1 hour.

Either soaking step could also include the addition of other compounds, e.g. H₂SO4, NH₃, in order to achieve higher performance later on in the process. However, it is preferred that acid, base or halogens not be used anywhere in the process or pre-treatment. The feedstock is preferably void of added sulfur, halogens, or nitrogen. The amount of sulfur, if present, in the composition is in the range of 0 to 1% by dry weight of the total composition. Additionally, the amount of total halogens, if present, are in the range of 0 to 1% by dry weight of the total composition. By keeping halogens from the feedstock, there are no halogens in the lignin conversion products.

The product comprising the first liquid is then passed to a separation step where the first liquid is separated from the soaked biomass. The liquid will not completely separate so that at least a portion of the liquid is separated, with preferably as much liquid as possible in an economic time frame. The liquid from this separation step is known as the first liquid stream comprising the first liquid. The first liquid will be the liquid used in the soaking, generally water and the soluble species of the feedstock. These water soluble species are glucan, xylan, galactan, arabinan, glucolygomers, xyloolygomers, galactolygomers and arabinolygomers. The solid biomass is called the first solid stream as it contains most, if not all, of the solids.

The separation of the liquid can again be done by known techniques and likely some which have yet to be invented. A preferred piece of equipment is a press, as a press will generate a liquid under high pressure.

The first solid stream is then steam exploded to create a steam exploded stream, comprising solids and a second liquid. Steam explosion is a well known technique in the biomass field and any of the systems available today and in the future are believed suitable for this step. The severity of the steam explosion is known in the literature as Ro, and is a function of time and temperature and is expressed as in the Experimental Section.

### Enzymatic Hydrolysis

After creation of the slurry, the slurry may be subjected to a catalyst composition, as described more fully below. It is in other words desirable to subject polysaccharide-containing biomasses to enzymatic hydrolysis in order to be able to subsequently produce bio-ethanol-containing fermentation broths suitable for distillation of ethanol.

As indicated above, the slurry containing the mechanically thermally treated ligno-cellulosic biomass can be subjected to enzymatic hydrolysis. The three major constituents in plant biomass are cellulose, hemicellulose and lignin, which are commonly referred to by the generic term lignocellulose. Cellulose, hemicellulose and lignin are present in varying amounts in different plants and in the different parts of the plant and they are intimately associated to form the structural framework of the plant.

Cellulose is a homopolysaccharide composed entirely of D-glucose linked together by β-1,4-glucosidic bonds and with a degree of polymerisation up to 10,000. The linear structure of cellulose enables the formation of both intra- and intermolecular hydrogen bonds, which results in the aggregation of cellulose chains into micro fibrils. Regions within the micro fibrils with high order are termed crystalline and less ordered regions are termed amorphous. The micro fibrils assemble into fibrils, which then form the cellulose fibres. The partly crystalline structure of cellulose along with the microfibrillar arrangement, gives cellulose high tensile strength, it makes cellulose insoluble in most solvents, and it is partly responsible for the resistance of cellulose against microbial degradation, i.e. enzymatic hydrolysis.

Hemicellulose is a complex heterogeneous polysaccharide composed of a number of monomer residues: D-glucose, D-galactose, D-mannose, D-xylose, L-arabinose, D-glucuronic acid and 4-0-methyl-D-glucuronic acid. Hemicellulose has a degree of polymerisation below 200, has side chains and may be acetylated. In softwood like fir, pine and spruce, galactoglucomaunan and arabino-4-methyl-glucuronoxylan are the major hemicellulose fractions. In hardwood like birch, poplar, aspen or oak, 4-O-acetyl-4-methyl-glucuronoxylan and glucomaunan are the main constituents of hemicellulose. Grasses like rice, wheat, oat and switch grass have hemicellulose composed of mainly glucuronoarabinoxylan.

Lignin is a complex network formed by polymerisation of phenyl propane units and it constitutes the most abundant non-polysaccharide fraction in lignocellulose. The three monomers in lignin are p-coumaryl alcohol, coniferyl alcohol and sinapyl alcohol, and they are most frequently joined through arylglyceryl-B-aryl ether bonds. Lignin is linked to hemicellulose and embeds the carbohydrates thereby offering protection against microbial and chemical degradation.

Bio-ethanol production from polysaccharide containing biomasses can be divided into three steps: 1) pretreatment 2) hydrolysis of the polysaccharides into fermentable carbohydrates 3) and fermentation of the carbohydrates.

Following the treatment, the next step in utilization of polysaccharide containing biomasses for production of bio-ethanol or other biochemicals is hydrolysis of the liberated starch. cellulose and hemicellulose into fermentable sugars. If done enzymatically this requires a large number of different enzymes with different modes of action. The enzymes can be added externally or microorganisms growing on the biomass may provide them.

The catalyst composition consists of the catalyst, the carrier, and other additives/ingredients used to introduce the catalyst to the process. As discussed above, the catalyst may comprise at least one enzyme or microorganism which converts at least one of the compounds in the biomass to a compound or compounds of lower molecular weight, down to, and including, the basic sugar or carbohydrate used to make the compound in the biomass. The enzymes capable of doing this for the various polysaccharides such as cellulose, hemicellulose, and starch are well known in the art and would include those not invented yet.

The catalyst composition may also comprise an inorganic acid preferably selected from the group consisting of sulfuric acid, hydrochloric acid, phosphoric acid, and the like, or mixtures thereof. The inorganic acid is believed useful for processing at temperatures greater than 100° C. The process may also be run specifically without the addition of an inorganic acid.

It is typical to add the catalyst to the process with a carrier, such as water or an organic based material. For mass balance purposes, the term catalyst composition therefore includes the catalyst(s) plus the carrier(s) used to add the catalyst(s) to the process. If a pH buffer is added with the catalyst, then it is part of the catalyst composition as well.

Cellulose is hydrolysed into glucose by the carbohydrolytic cellulases. The prevalent understanding of the cellulolytic system divides the cellulases into three classes; exo-1,4-β-D-glucanases or cellobiohydrolases (CBH) (EC 3.2.1.91), which cleave off cellobiose units from the ends of cellulose chains; endo-1,4-β-D-glucanases (EG) (EC 3.2.1.4), which hydrolyse internal β-1,4-glucosidic bonds randomly in the cellulose chain; 1,4-β-D-glucosidase (EC 3.2.1.21), which hydrolyses cellobiose to glucose and also cleaves of glucose units from cellooligosaccharides.

The different sugars in hemicellulose are liberated by the hemicellulases. The hemicellulytic system is more complex than the cellulolytic system due to the heterologous nature of hemicellulose. The system involves among others endo-1,4-p-D-xylanases (EC 3.2.1.8), which hydrolyse internal bonds in the xylan chain; 1,4-p-D-xylosidases (EC 3.2.1.37), which attack xylooligosaccharides from the non-reducing end and liberate xylose; endo-1 sl-p-Dvmannanases (EC 3.2.1.78), which cleave internal bonds; 1,4-β-D-maImosidases (EC 3.2.1.25), which cleave mannooligosaccharides to mannose. The side groups are removed by a number of enzymes; α-D-galactosidases (EC 3.2.1.22), α-L-arabinofuranosidases (EC 3.2.1.55), α-D-glucuronidases (EC 3.2.1.139), cinnamoyl esterases (EC 3.1.1.-), acetyl xylan esterases (EC 3.1.1.6) and feruloyl esterases (EC 3.1.1.73).

In combination with pre-treatment and/or enzymatic hydrolysis of lignocellulosic biomasses, it has been found that the use of oxidative enzymes can have a positive effect on the overall hydrolysis as well as the viability of the microorganisms employed for e.g. subsequent fermentation. The reason for this effect is the oxidative crosslinking of lignins and other phenolic inhibitors as caused by the oxidative enzymes. Typically laccase (EC 1.10.3.2) or peroxidase (EC 1.1 1.1.7) are employed either externally or by incorporation of a laccase gene in the applied microorganism.

Because the ligno-cellulosic biomass may contain starch, the important enzymes for use in starch hydrolysis are alpha-amylases (1,4-α-D-glucan glucanohydrolases, (EC 3.2.1.1)). These are endo-acting hydrolases which cleave 1,4-α-D-glucosidic bonds and can bypass but cannot hydrolyse 1,6-α-D-glucosidic branchpoints. However, also exo-acting glycoamylases such as beta-amylase (EC 3.2.1.2) and pullulanase (EC 3.2.1.41) can be used for starch hydrolysis. The result of starch hydrolysis is primarily glucose, maltose, maltotriose, α-dextrin and varying amounts of oligosaccharides. When the starch-based hydrolysate is used for fermentation it can be advantageous to add proteolytic enzymes. Such enzymes may prevent flocculation of the microorganism and may generate amino acids available to the microorganism. Therefore, if the biomass contains starch, then glucose, maltose, maltotriose, α-dextrin and oligosaccharides are examples of a water soluble hydrolyzed species obtainable from the hydrolysis of the starch containing biomass and the afore mentioned alpha-amylases are specific examples, as well as those mentioned in the experimental section, of catalysts for the hydrolysis of starch.

These above embodiments are not designed to limit the specification or claims, as there are many configurations available to one of ordinary skill, which include a series of continuous vessels, or semi batch reactors or in combination with or without plug flow reactors.

The hydrolyzed composition comprises water and simple sugar or sugars, and optionally soluble oligomeric sugars which are not metabolized by the microbial organism. It is known in the art that simple oligomeric sugars, such as dimers, may be metabolized by microorganisms. The hydrolyzed composition further comprises water insoluble xylans, glucans and lignin. At least a portion of the lignin and water insoluble sugars may be removed from the hydrolyzed composition, for instance by means of a filter, prior to the cultivation of the microbial organism in the presence of the hydrolyzed composition.

The hydrolyzed composition is noted as very specific, in that one or any combination of the following improvements are achieved:

A) the levels of inhibitors and undesirable products, which are important in primis for the microbial organism growth, as well as for fermentation and final product separation, are much lower than those obtained by other treatments disclosed in the prior art;
B) the global sugars solubilization yield is higher than other process;
C) the biomass de-structuring is improved with respect to other treatments.

To avoid dilution effects, the hydrolyzed composition may be characterized in terms of the ratio of the amount the inhibitory compounds to the total amount of simple sugars in the hydrolyzed composition. The hydrolyzed composition of the disclosed method is characterized by ratios of the amount the inhibitory compounds to the total amount of simple sugars of the hydrolyzed composition lower than the corresponding ratios of ligno-cellulosic hydrolyzates obtained by other treatments.

In a preferred embodiment, the thermally treated ligno-cellulosic biomass is subjected to a fiber shives reduction step. As the fiber shives reduction increases the glucans accessibility of the thermally treated ligno-cellulosic biomass without altering its chemical composition, specifically without producing degradation products which act as inhibitory compounds, the hydrolyzed composition obtained from the thermally treated ligno-cellulosic biomass which has been subjected to fiber shives reduction will be characterized by improved ratio of the amount the inhibitory compounds to the total amount of simple sugars.

In particular:
the ratio of the amount of acetic acid to the total amount of simple sugars of the hydrolyzed composition may be less than 0.15, preferably less than 0.10, more preferably less than 0.05, even more preferably less than 0.02 and most preferably less than 0.01;
the ratio of the amount of furfural to the total amount of simple sugars of the first hydrolyzed composition may be less than 0.01, preferably less than 0.005, more preferably less than 0.001, even more preferably less than 0.0005 and most preferably less than 0.0003;
the ratio of the amount of 5HMF to the total amount of simple sugars of the first hydrolyzed composition may be less than 0.02, preferably less than 0.01, more preferably less than 0.005, even more preferably less than 0.001 and most preferably less than 0.0005.

### Microbial Organism Cultivation

In the present specification, the terms “microbial,” “microbial organism” or “microorganism” are equivalent terms for indicating any organism that exists as a microscopic cell included within the domains of archaea, bacteria or eukarya. Therefore, the term comprises prokaryotic or eukaryotic cells or organisms having a microscopic size and includes bacteria, archaea and eubacteria of all species as well as eukaryotic microorganisms such as yeast and fungi. The term also includes cells of any species that can be cultured for the production of a biochemical.

A preferred microbial organism is a yeast. According to K. A. Jacques, T. P. Lyons, D. R. Kelsall, The Alcohol Textbook, 4th Edition, p. 85 ‘yeast is a fungus where the unicellular form is the most predominant and which reproduces by budding (or fission)’.

The yeast of the present disclosure can be selected from any known genus and species of yeasts. Yeasts are described for example by N. J. W. Kreger-van Rij, “The Yeasts,” Vol. 1 of Biology of Yeasts, Ch. 2, A. H. Rose and J. S. Harrison, Eds. Academic Press, London, 1987. In one embodiment the yeast is selected from the group consisting of Saccharomyces, Zygosaccharomyces, Candida, Hansenula, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, and Schwanniomyces. Preferably the yeast is selected from Saccharomyces cerevisaie.

The yeast maybe haploid or diploid.

In another embodiment, the microbial organism is a bacterium. Preferably, the bacterium is selected from the group consisting of Escherichia coli, Klebsiella oxytoca, Anaerobiospirillum succiniciprodiicens, Actino bacillus succinogenes, Mannheimia succiniciprodiicens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida.

The microbial organism may be a wild-type microbial organism or a recombinant microbial organism. In the present disclosure, “wild-type” and “naturally-occurring” are equivalent terms, as well as “recombinant” and “non-naturally occurring”.

The term “wild-type microbial organism” describes a microbial organism that occurs in nature, i.e. a microbial organism that has not been genetically modified. A wild-type microorganism can be genetically modified to express or overexpress a first target enzyme.

The term “non-naturally occurring” microbial organism means that the microbial organism has at least one genetic alteration not normally found in a naturally occurring strain of the referenced species, including naturally-occurring strains of the referenced species. Genetic alterations may include, for example, modifications introducing expressible nucleic acids encoding metabolic polypeptides, other nucleic acid additions, nucleic acid deletions and/or other functional disruption of the microbial organism's genetic material. Such modifications may include, for example, coding regions and functional fragments thereof, for heterologous, homologous or both heterologous and homologous polypeptides for the referenced species. Additional modifications may include, for example, non-coding regulatory regions in which the modifications alter expression of a gene or operon.

The non-naturally occurring microbial organism of the present disclosure can contain stable genetic alterations, which refers to microbial organism that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

Such genetic alterations include, for example, genetic alterations of species homologs, in general, and in particular, orthologs, paralogs or nonorthologous gene displacements.

The non-naturally occurring microbial organism strain can be prepared by methods known in the art and methods yet to be disclosed, including those involving homologous recombination, directed mutagenesis or random mutagenesis, among others. In certain cases, the recombinant microbial organism can be recovered by a process involving natural selection. A review of the main methods may be found in Sambrook et al., Molecular Genetics: A Laboratory Manual, Cold Spring Harbor Laboratory Press, which provides further information regarding various techniques known in the art.

The microbial organism is grown by cultivating the microbial organism in the presence of the hydrolyzed composition disclosed in the present specification. The simple sugars in the hydrolyzed composition are a preferred carbon source converted to microbial organism biomass and the cultivation conditions are selected in such a way that microbial organism growth effectively occurs. The cultivation environment may further comprise other components, such as simple sugars different from those in the first hydrolyzed composition, a nitrogen source and additional salts required for microorganism metabolism. Optionally added simple sugars may be contained or extracted for instance from molasses.

Depending also on aeration conditions, nutrients and carbon source concentration in the cultivation environment, during the cultivation the growth of microbial organism may occur simultaneously to the conversion of the carbon source to useful organic compounds, such as ethanol in the case of yeast. The process of conversion of a carbon source to an organic compound is generically indicated as fermentation.

The cultivation of the microbial organism may be conducted in different cultivation configurations known in the art, such as batch, fed-batch, repeated fed-batch, chemostat or continuous configurations. An exemplary description of continuous cultivation may be found in K. A. Jacques, T. P. Lyons, D. R. Kelsall, The Alcohol Textbook, 4th Edition, p. 132-140.

Batch cultivation refers to the cultivation of the microbial organism with growth occurring in a substantially fixed volume of the cultivation environment that is continually being altered by the actions of the growing yeasts until it is no longer suitable for growth. In batch culture, all components of the cultivation environment required for the growth of the microbial organism are present in the cultivation environment before beginning cultivation, except for oxygen in aerobic cultivation.

Fed-batch cultivation refers to a cultivation technique in which one or more components of the cultivation environment are added into the cultivation environment over the course of cultivation of the microbial organism. In contrast to a chemostat cultivation, the microbial organism is contained in the cultivation environment during cultivation. In some cases, all nutrients are gradually fed to the cultivation environment. The time conditions, temperature conditions, pH conditions, aeration conditions, and the rate at which certain nutrients are fed to the cultivation environment depend on the particular microbial organism that is being used.

Chemostat cultivation refers to a cultivation technique in which one or more components of the cultivation environment are added into the cultivation environment and a fraction of the microbial organism biomass may be removed from the cultivation environment during cultivation. In this case, both specific growth rate and cell number can be controlled independently. A chemostat allows control of both the cell density and the microbial organism growth in the cultivation environment through dilution rate and alteration of the concentration of a limiting nutrient, such as a carbon or nitrogen source.

In a preferred embodiment, the microbial organism is grown by feeding the microbial organism exclusively with the disclosed hydrolyzed composition, more specifically with the simple sugars comprised in the hydrolyzed composition. No simple sugar or sugars coming from other carbon sources, such as for instance molasse or synthetic sugars, are added to the cultivation environment.

In another embodiment, the microbial organism is grown by feeding the microbial organism with the hydrolyzed composition under added simple sugar conditions of having an amount of optionally added simple sugar or sugars in the range of 0 to 30% by weight of the simple sugars of the hydrolyzed composition for a portion of the cultivation time which is less than 70% of the cultivation time. Optionally added simple sugar or sugars are a carbon source different from the disclosed hydrolyzed composition. Molasse is a preferred carbon source of optionally added simple sugar or sugars.

The cultivation time is the amount of time measured from the addition of the initial microbial organism amount, or inoculum, to the cultivation environment to the harvest, removal, or separation of microbial organism biomass from the cultivation environment. In the case of multiple removals, the cultivation time ends at the time when the last removal of the microbial organism biomass is ended.

The cultivation time may be less than 36 hours, preferably less than 24 hours, more preferably less than 18 hours, even more preferably less than 12 hours, most preferably less than 6 hours.

The expression “added simple sugar conditions” means generally that more than 50% by weight of the microbial organism feed is from the simple sugars in the hydrolyzed composition and not from added simple sugars. An exemplary added simple sugar condition is when the amount of optional simple sugars added to the process, if any is added at all, is in the range of 0 to 30% by weight of the simple sugars in the hydrolyzed composition. More preferably, the optional simple sugars added should be in the range of 0 to 10% by weight of the simple sugars in the hydrolyzed composition, with 0 to 5% by weight being even more preferred, with 0 to 2.0% being the most preferred. Additionally, the phrase simple sugars added means that there could be one or more simple sugars added.

The added simple sugar conditions should be maintained for less than a portion of the cultivation time. Expressed quantitatively, the added simple sugar conditions should be maintained for less than 60% of the cultivation time, with 50% being more preferred, 40% being even more preferred, with 30% being even yet more preferred with 20%, 10% and 5% of the cultivation time being the most preferred.

The hydrolyzed composition is a carbon source and may be added to the cultivation environment together with a carbon source, such as molasse, but may also be added separately from the carbon source. According to the invention the hydrolyzed composition may be added to the cultivation environment either prior to inoculation, simultaneously with inoculation or after inoculation of the initial amount of microbial organism into the cultivation environment in an amount at least corresponding to the amount of simple sugars needed to grow the microbial organism. When the hydrolyzed composition is added during the cultivation time, a new calculation of the amount of optional simple sugars added or the ratio of optional simple sugars to the simple sugars in the hydrolyzed composition is done. While the amount of simple sugars may not have been low enough during the initial part of the cultivation time, by adding the hydrolyzed composition to the cultivation environment, the amount of optional simple sugars added would fall within the specified ranges, at least for the time remaining in the cultivation time.

A person skilled in the art can easily determine when to add and what amount of the hydrolyzed composition according to the invention. During the time span of cultivation the simple sugars of the hydrolyzed composition are normally consumed by the microbial organism and kept within the previously specified limits.

As mentioned above the hydrolyzed composition is used the same way a carbon source, such as glucose, is normally used in well-known microbial organism cultivation processes.

The total amount of simple sugars in the cultivation environment is the sum of the amount of simple sugars of the hydrolyzed composition and the amount of optionally added simple sugar or simple sugars to the cultivation environment. The total amount of simple sugars in the cultivation environment may be kept constant or may be varied during cultivation time, depending on the feed rate and cultivation conditions.

In one embodiment, the concentration of the total amount of simple sugars in the cultivation environment is lower than 200 g/l, preferably lower than 100 g/l, more preferably lower than 50 g/l, even more preferred lower than 30 g/l, most preferred lower than 20 g/l for a portion of the cultivation time which is at least 50% of the cultivation time.

Preferably, the microbial microorganism is cultivated in aerobic condition of having dissolved oxygen in the cultivation environment in sufficient amount to promote effectively the growth of the microbial organism during the cultivation time. In the case of a yeast, preferably the dissolved oxygen concentration is comprised in a range from 4 ppm to 60 ppm of dissolved oxygen.

The aerobic condition may be obtained by aerating the cultivation environment with molecular oxygen or a mixture of gases comprising molecular oxygen, such as air. Aeration may be obtained by vigorous agitation of the cultivation environment in an atmosphere comprising molecular oxygen. Aerobic condition comprises also micro-aerobic condition of having an oxygen concentration in the atmosphere greater than zero but less than that in open air. Preferably aeration is obtained by injecting molecular oxygen or air in the cultivation environment, by means of techniques and air flow configurations well known in the art. In one embodiment, the cultivation occurs in an air flow less than 1VVm, preferably less than 10VVh, more preferably less than 5VVh, even more preferably less than 1VVh, yet even more preferably less 0.5VVh, being less than 0.1VVh and less than 0.05 VVh the most preferred conditions. 1VVh and 1 VVm correspond to the flow of an air volume equal to the cultivation environment volume per hour and per minute respectively. It is important to remember that the amount of air or oxygen injected into the cultivation medium may bear little relationship to the amount of oxygen that actually dissolves. Thus it is necessary to measure the oxygen in solution in order to know what is available to the microbial organism.

Thus, according to one aspect, the invention relates to processes of growing a microbial organism comprising cultivating said microbial organism under conditions conducive for the growth of the microbial organism. Such conditions comprise a set of physical parameters, such as temperature, and chemical parameters, such as pH, which are defined according to growth requirements of the specific microbial organism.

After cultivation, the microbial organism may optionally be recovered using methods well known in the art. For example, the recovery from the cultivation environment may be done using conventional procedures including, but not limited to, centrifugation, filtration, extraction, or precipitation.

The cultivated microbial organism may be used for fermenting at least a portion of a second hydrolyzed composition, which comprises simple sugars and complex sugars. Preferably, the first hydrolyzed composition and the second hydrolyzed composition are obtained by means of the disclosed treatment from the same ligno-cellulosic feedstock. In the fermentation of the second hydrolyzed composition, simple sugars are converted to organic compounds, such as ethanol in the case of yeast, preferably in anaerobic condition. Even if all the simple sugars of the second hydrolyzed composition may be subjected to fermentation, in a preferred embodiment a first portion of the simple sugars of the second hydrolyzed composition are converted to new microbial organism biomass, simultaneously to the fermentation of a second portion of the simple sugars of the second hydrolyzed composition.

In another embodiment, the second hydrolyzed composition is subjected to Simultaneous Saccharification and Fermentation (SSF), in which at least a portion of the complex sugars of the second hydrolyzed composition are hydrolyzed to simple sugars in the presence of a second catalyst, which preferably is an enzyme or enzyme cocktail, and simultaneously the simple sugars of the second hydrolyzed composition are fermented to organic compounds, preferably ethanol. Simultaneous Saccharification and Fermentation is a process well known in the art.

In a preferred embodiment, the microbial organism is a yeast and the ligno-cellulosic feedstock is subjected to the disclosed treatment to produce a pre-treated composition. The pre-treated composition is inserted in a first vessel and subjected to enzymatic hydrolysis under conditions conducive to produce the hydrolyzed composition. A portion of the hydrolyzed composition is removed from the first vessel and inserted into a second vessel. Preferably, the removed portion contains no, or few, lignin and water insoluble sugars of the hydrolyzed composition. In the second vessel, yeast is cultivated under conditions conducive to the growth of the yeast. During the yeast growth, a certain amount of ethanol may also be produced, depending on growth conditions. The grown yeast and the portion of the hydrolyzed composition not used for the yeast cultivation are inserted into a third vessel. The yeast may be separated from the cultivation environment or, preferably, all the cultivation environment comprising the yeast is inserted in the third vessel. In the third vessel, fermentation of simple sugars to ethanol is conducted. During fermentation, also yeast may be grown, depending on fermentation conditions. Preferably, in the third vessel fermentation is conducted simultaneously with the hydrolysis of complex sugars of the hydrolyzed composition.

### EXPERIMENTAL

### Preparation of Thermally Treated Ligno-Cellulosic Biomass

Wheat straw was used as the ligno-cellulosic biomass feedstock.

Wheat straw was subjected to a thermal treatment composed of a soaking step followed by a steam explosion step according to the following procedure.

Ligno-cellulosic biomass was introduced into a continuous reactor and subjected to a soaking treatment. The soaked mixture was separated into a soaked liquid and a fraction containing the solid soaked raw material by means of a press. The fraction containing the solid soaked raw material was subjected to steam explosion. Steam exploded products were separated into a steam explosion liquid and a steam exploded solid. Steam exploded solid is the exemplary thermally treated ligno-cellulosic biomass before fiber shives reduction used in the present experimental section and they are indicated by the -BSR (Before fiber Shives Reduction) extension following the sample code.

Pretreatment parameters of the ligno-cellulosic biomass are reported in Table 1.

Severity of each thermal treatment step R₀₁ and R₀₂ was calculated according the formula:

R₀₁=log₁₀(Q₁), wherein

Q₁=t₁exp((T₁−100)/14.75)

R₀₂=log₁₀(Q₂), wherein

Q₂=t₂exp((T₂−100)/14.75),

wherein time t₁ and t₂ is measured in minutes and temperature T₁ and T₂ is measured in Celsius.

The total severity factor R₀ was calculated according to the formula:

R₀=log₁₀(Q₁+Q₂)

TABLE 1
Process parameters used in the thermal treatment

| | Soaking | | Steam explosion | | | | |
|---|---|---|---|---|---|---|---|
| | Temper- | Time | Temper- | Time | | | |
| | ature | (min- | ature | (min- | | | |
| Sample | (° C.) | utes) | (° C.) | utes) | R₀₁ | R₀₂ | R₀ |
| S01-BSR | 155 | 65 | 180 | 2 | 3.43 | 2.66 | 3.50 |
| S02-BSR | 155 | 65 | 195 | 2 | 3.43 | 3.10 | 3.60 |
| S03-BSR | 155 | 65 | 187 | 8 | 3.43 | 3.46 | 3.75 |
| S04-BSR | 155 | 65 | 195 | 4 | 3.43 | 3.40 | 3.72 |
| S05-BSR | 155 | 65 | 202 | 8 | 3.43 | 3.91 | 4.03 |
| S06-BSR | 155 | 65 | 210 | 16 | 3.43 | 4.44 | 4.48 |
| S07-BSR | 158 | 65 | 201.5 | 4 | 3.52 | 3.59 | 3.86 |
| S08-BSR | 158 | 65 | 202.5 | 2 | 3.52 | 3.32 | 3.73 |

### Fiber Shives Reduction of the Thermally Treated Ligno-Cellulosic Biomass

All the thermally treated ligno-cellulosic biomass were subjected to a fiber shives reduction step by means of a counter-rotating twin screw extruder (Welding Engineers Inc., model HTR 30 MM (HTR 30.22.22.22.13.E1), Blue Bell, PA.), barrel length to screw diameter ratio of 54:1. The machine was fitted to a 25-hp motor, which has a provision to adjust the screw speed from 0 to 500 rpm. The parameters of the profile of the screws are reported in FIG. 1.

The thermally treated ligno-cellulosic biomass was treated at 250 rpm to reduce fiber shives. The thermally treated ligno-cellulosic biomass was inserted in the extruder at a temperature of 25° C. The thermally treated ligno-cellulosic biomass exited the extruder as a solid at about 25° C. The thermally treated ligno-cellulosic biomass was inserted manually in the extruder at an inlet rate of approximately 5 Kg/h on wet basis, at a moisture content of about 60%. Residence time was estimated be to approximately 3 minutes.

The specific energy consumption for fiber shives reducing a Kg of thermally treated ligno-cellulosic biomass was evaluated by the equation:

SEC=Absorbed power/T,

wherein Absorbed power is measured in W, T is the material throughput, in Kg/h and SEC is measured in Wh/Kg.

The absorbed power is the electrical power absorbed by the electrical engine of the extruder. Thereby, the SEC parameter is an overestimation of the specific mechanical energy (SME), which is a parameter often reported in the prior art and is the mechanical energy applied to the thermally pretreated ligno-cellulosic biomass (see for example Wen-Hua Chen et al., Bioresource Technology 102 (2011), p. 10451).

The SEC was evaluated to be in the range of 0.1-0.2 kWh/Kg of thermally treated ligno-cellulosic biomass on wet basis. The specific energy consumption is much lower that the specific energy reported in the prior art, as for example in WO2011044292A2, wherein an energy of 1.03 kWh/kg is used.

The extruded thermally treated ligno-cellulosic biomass for reducing fiber shives is the exemplary thermally treated ligno-cellulosic biomass after fiber shives reduction used in the following examples and are indicated by the -ASR (After fiber Shives Reduction) extension following the sample code.

### Composition

Composition of materials was determined according to standard analytical methods listed at the end of the experimental section to quantify soluble sugars (glucose, xylose, glucooligomers and xylooligomers), insoluble sugars (glucans and xylans), xylans degradation products (furans, such as furfural), glucans degradation products (HMF), and lignin and other compounds. The compositions of corresponding BSR and ASR materials were identical within the measurement error and only ASR compositions of exemplary samples (S01 to S06) are reported in Table 2. Results are reported in terms of weight percent of the dry matter of the samples. It is noted that the percent amount of glucans and xylans degradation products is negligible or very low, namely less than 1% in all the samples, thanks to the low severity of the thermal treatment. Acetic acid is produced as an effect of the thermal treatment on the acetyl groups in the ligno-cellulosic biomass and it is considered an enzyme inhibitory compound, but not a sugar degradation product which potentially limits the yield of the process. Also the content of acetic acid is negligible. It is noted that the percent ratio of insoluble xylans to insoluble glucans decreases with severity factor R02, as the thermal treatment removes preferentially xylans.

TABLE 2
Composition of thermally treated biomass
after fiber shives reduction.

| Composition, | | | | | | |
|---|---|---|---|---|---|---|
| % wt. DB | S1-ASR | S2-ASR | S3-ASR | S4-ASR | S5-ASR | S6-ASR |
| Glucose | 0 | 0 | 0 | 0 | 0.101 | 0.088 |
| Xylose | 0 | 0.244 | 0 | 0.8 | 1.734 | 1.546 |
| Glucolygomers | 0 | 0.258 | 0 | 0.556 | 0.77 | 0.731 |
| Xylolygomers | 0 | 3.849 | 0 | 4.886 | 2.112 | 2.634 |
| Insoluble | 43.658 | 46.392 | 50.271 | 47.844 | 42.705 | 44.394 |
| glucans | | | | | | |
| Insoluble | 13.498 | 14.637 | 13.046 | 11.122 | 3.994 | 3.79 |
| xylans | | | | | | |
| Lignin | 20.685 | 22.498 | 23.225 | 22.61 | 21.34 | 22.723 |
| Others | 22.159 | 11.933 | 13.458 | 11.945 | 26.656 | 23.351 |
| Furfural | 0 | 0.007 | 0 | 0.024 | 0.057 | 0.08 |
| HMF | 0 | 0.024 | 0 | 0.043 | 0.119 | 0.142 |
| Acetic Acid | 0 | 0.158 | 0 | 0.17 | 0.412 | 0.521 |
| Insoluble | 0.309 | 0.316 | 0.26 | 0.232 | 0.094 | 0.085 |
| xylans/ | | | | | | |
| insoluble | | | | | | |
| glucans | | | | | | |
| Insoluble | 2.11 | 2.06 | 2.16 | 2.12 | 2.00 | 1.95 |
| glucans/ | | | | | | |
| lignin | | | | | | |

### Glucose/Xylose Recovery and Glucans Accessibility

Glucose recovery is the percent ratio between the total amount of glucans in the thermally treated biomass before fiber shives reduction (as glucose equivalent calculated including insoluble glucans, gluco-oligomers, cellobiose and glucose present in both solid and liquid streams) and the amount of glucans (converted in glucose equivalent) present in the raw material before the thermally treatment. The complementary to 100% of the glucose recovery represent therefore the total amount of glucans degradation products as an effect of the thermal treatment.

Xylose recovery is the percent ratio between the total amount of xylans in the thermally treated biomass before fiber shives reduction (as xylose equivalent calculated including insoluble xylans, xylo-oligomers, xilobiose and xylose present in both solid and liquid streams) and the amount of xylans (converted in xylose equivalent) present in the raw material before the thermal treatment. The complementary to 100% of the xylose recovery represents therefore the total amount of xylans degradation products as an effect of the thermal treatment.

Glucans accessibility is defined as the percent amount of insoluble glucans enzymatically hydrolyzed to soluble compounds with respect to the amount of insoluble glucans in the pre-treated materials (before and after fiber shives reduction) and calculated as (1−% insoluble glucans at the end of the hydrolysis)/(% insoluble glucans at the beginning of the hydrolysis), when hydrolysis is conducted in excess of enzymes and for a long time. Glucans accessibility was determined according to the following procedure.

Pretreated material was mixed with water in a volume of 1500 ml to obtain a mixture having a 7.5% dry matter content and the mixture was inserted into an enzymatic reactor. pH was set to 5.2 and temperature was set to 50° C. An enzyme cocktail (CTec3 by Novozymes) was added, corresponding to a concentration of 26 g of cocktail solution per 100 gram of glucans contained in the mixture.

Enzymatic hydrolysis was carried out for 48 hours under agitation. The content of glucans, glucose and glucooligomers in the mixture was measured at different times of the enzymatic hydrolysis.

Glucans accessibility and xylose and glucose recovery was determined for all the BSR and ASR materials.

In FIG. 2 the glucans accessibility and in FIG. 3 the xylose and glucose recovery in function of R₀₂ are reported. All the plots in this experimental section are reported in function of R₀₂, as this severity factor is related to the steam explosion effect. Similar considerations hold in the case that R₀ is considered as the independent variable in the graphs.

It is noted that glucans accessibility of BSR material increases by increasing severity factor, but a bigger amount of xylans are degraded. The fiber shives reduction treatment is effective to increase the glucans accessibility at low severity factor, without degrading xylans (or degrading a very few amount of) to degradation products. Thereby, also at low severity factor, a glucans accessibility greater than 90% is obtained. Increasing the severity factor, the effectiveness of the fiber shives reduction treatment on glucans accessibility is less pronounced.

In the case of glucans recovery, the degradation effect is less pronounced but the effects of thermal and fiber shives reduction treatment are similar to those observed for xylans recovery.

### Automated Optical Analyses

The samples were analyzed by automated optical analysis, using unpolarized light for determining fibres, fines and fiber shives content, as well as length and width. ISO 16065 2:2007 protocol was used in fibres analyses.

The instrument used was a MorFi analyser from Techpap, Grenoble, France.

Briefly, 2 g of air dried sample was disintegrated in a low consistency pulper for 2000 revolutions in approximately 2 litres of tap water, thus reaching a stock concentration of about 1 g/l.

The suspension was stirred very well before withdrawing the sample to perform the measurement according to the manufacturer's instructions. Each sample was run in duplicate or in triplicate in case of higher standard deviation.

According to Morfi analysis software, the treated ligno-cellulosic biomass is composed by:

Fiber shives: elements having a width greater than 75 micron
Fibres: elements having a width equal to or less than 75 micron and a length greater than 200 micron
Fines: having a width equal to or less than 75 micron and a length less than 200 micron

The width of the fibres, fines and fibers shives remained substantially unchanged after the fiber shives reduction treatment.

In the graphs of FIG. 4 it is reported the area-weighted distribution of fibres and fines length of BSR and ASR materials produced at low severity factor (S02-BSR and S02-ASR, FIG. 4a) and high severity factor (S05-BSR and S05-ASR, FIG. 4b) relative to all the sample. Briefly, the percent area value of each length class has been calculated as percent ratio of the sum of the area of all the fibres and fines in each length class and the sum of the area of all the fines, fibres, and fiber shives.

It is noted that S05-BSR has a greater percent area of fines and a lower percent area of long fibres with respect to S02-BSR, as expected considering the higher severity of S05-BSR thermal treatment. This corresponds to a higher glucans accessibility of S05-BSR (about 95%) with respect to S02-BSR (84%).

The fiber shive reduction treatment reduces the percent area of long fibres (or equivalently the number of long fibres) and increases the population of fines and short fibres in both the samples, but:
the reduction of the percent area of long fibres in S05-ASR, with respect to S05-BSR, is similar to the corresponding reduction in S02-ASR;
the percent area of fines in S05-ASR is greater than in S02-ASR;
despite the fact that S05-ASR contains more fines/short fibres than S05-BSR (in other words, it is more refined), the accessibility is within the experimental error (93% and 94%);
despite the fact that S05-ASR contains more fines/short fibres than S02-ASR, the corresponding accessibility are very close (93% and 92% respectively).

In the graph of FIG. 5 it is reported the area-weighted distribution of fiber shives of S02-BSR (FIG. 5a) and S05-BSR (FIG. 5b) and related ASR materials. The percent area value of each length class has been calculated as percent ratio of the sum of the area of all the fiber shives in each length class to the sum of the areas of all the fines, fibres, and fiber shives.

It is highlighted that:
S05-BSR has a lower percent area of shives than S02-BSR, in particular shives longer than about 737 μm, evidencing that that steam explosion is effective in reducing big shives;
the percent area of shives is strongly reduced by the mechanical treatment in S02-BSR, due to the large starting shives population.
the accessibility of S02-BSR is strongly enhanced by the reduction in long shives population;
The accessibility of S05-BSR is not affected by the fiber shives reduction treatment because the limited percent area of long shives.

In the graph of FIG. 6 it is reported the percent area of all the shives having a length greater than 737 μm in function of the second severity cooking R₀₂ of exemplary samples before and after fiber shives reduction. S06-BSR was produced at the maximum severity factor of R₀₂ of 4.44 sufficient to remove substantially all shives. The percent area of all the shives having a length greater than 737 μm has been calculated as the percent ratio of the sum of the areas of all the shives and the sum of the areas of all the fines, fibres, and fiber shives.

These results highlight the fact that the increase in glucans accessibility is not strictly related to fibre size reduction, that is, once the fibres are accessible to the enzyme, any further decrease in fibre length is not effective on enzymatic accessibility of the fibre, thereby energy is spent without obtaining any beneficial effect on accessibility.

Instead, experiments show that it is the reduction of the amount of fiber shives to be effective on the enzymatic accessibility, depending clearly from the starting population of fiber shives. If the thermal treatment is performed at a severity high enough to produce a thermally treated material having a low amount of fiber shives, more specifically of long fiber shives, the fiber shives reduction treatment has not effect on the accessibility of the material. Unfortunately, such a high severity thermal treatment degrade a relevant amount of glucans and xylans to detrimental degradation products.

Basically, the experiments highlight that fiber shives are fiber bundles which are not accessible to the enzymes, thereby limiting the glucans accessibility, and that the fiber shives reduction treatment is useful when it convert fiber shives to fibres. As a consequence, the combination of the thermal treatment in mild conditions and the treatment to reduce the amount of fiber shives increases the glucans accessibility and xylose recovery without degrading a significant amount of sugars in the ligno-cellulosic biomass.

### Torque Measurement of Slurried Samples

Torque measurement experiments were run in a cylindrical vessel whose characteristics are here reported.

D (diameter)=105 mm
H (height)=145 mm

The reactor is fitted with a stirrer tool IKA R 1375 to give the following configurations:

D (stirrer width)=70 mm
D (stirrer height)=70 mm
H (stirrer distance from the vessel bottom)=10 mm
Agitation was provided by IKA Eurostar 60 control motors (power: 126 W).

With no material inserted, the no load torque at 50 rpm was 0 N cm. An amount of material corresponding to 80 gr on dry basis was inserted in the vessel and water was added to reach a dry matter of 20%.

The mixture was agitated at 50 rpm for 10 seconds. The torque value of each run was calculated as the mean of the maximum and minimum value during 5 seconds measuring time.

The measurement was replicated three times and the torque was calculated as the mean value of the three runs.

After each torque measurement at a fixed dry matter, the dry matter was reduced to 18%, 16%, 14%, 12%, 10%, 8% by subsequent addition of water. Temperature was maintained to 25° C.

In table 3 torque values of exemplary samples, collected at different dry matter, are reported. Values below the sensitivity of the measurements are reported as 0.

TABLE 3
Torque measurements of samples at different dry matter
Torque, N*cm

| DM, | S01- | S01- | S02- | S02- | S03- | S03- | S04- | S04- | S05- | S05- |
|---|---|---|---|---|---|---|---|---|---|---|
| % | BSR | ASR | BSR | ASR | BSR | ASR | BSR | ASR | BSR | ASR |
| 20% | 87 | 11 | 49 | 8 | 59 | 17 | 36 | 2 | 0 | 0 |
| 18% | 54 | 7 | 40 | 6 | 45 | 10 | 20 | 1 | 0 | 0 |
| 16% | 43 | 5 | 31 | 3 | 31 | 8 | 13 | 0 | 0 | 0 |
| 14% | 25 | 5 | 19 | 2 | 17 | 5 | 8 | 0 | 0 | 0 |
| 12% | 17 | 3 | 10 | 1 | 11 | 3 | 5 | 0 | 0 | 0 |
| 10% | 9 | 1 | 6 | 0 | 8 | 1 | 1 | 0 | 0 | 0 |
| 8% | 3 | 0 | 1 | 0 | 3 | 0 | 0 | 0 | 0 | 0 |

In FIG. 7 the torque of S01 and S04 samples (BSR and corresponding ASR materials), measured at different dry matter, are plotted as an example.

In FIG. 8 the torque measured at 18% dry matter as a function of the severity factor is reported.

It is noted that at fixed dry matter the torque values decreases by increasing the thermal treatment severity factor and that samples thermally treated at the highest severity factor present a torque value which is very small—or zero—even at the high dry matter values. Torque values are dependent from the experimental setup and procedure used, but they are directly related to viscosity measurements. Thereby, viscosity strongly decrease increasing the severity factor of the thermal treatment.

By applying the disclosed fiber shives reduction treatment to the thermally treated samples, the torque values at each dry matter decrease and this effect is enhanced at low severity.

Thereby, the combination of the thermal treatment in mild conditions and the treatment to reduce the amount of fibers shives of the thermally treated biomass strongly reduces the torque/viscosity of a slurry of the corresponding thermally treated biomass after fiber shives reduction. Again, this is obtained without degrading significant amount of sugars of the ligno-cellulosic biomass.

As reported in following experimental sections, the torque/viscosity values of the slurry prepared using the thermally treated ligno-cellulosic biomass after shives reduction are comparable to the torque/viscosity values of corresponding thermally treated biomass before fiber shives reduction which have been enzymatically hydrolyzed.

### Saturation Humidity

Saturation humidity is the maximum amount of water that could be absorbed by the ligno-cellulosic biomass. The water added to the material after the material has reached its saturation humidity value is not entrapped into the solid material and will be present as free water outside the solid. Material properties evaluated using the saturation humidity procedure are equivalent to those given by the well-known in the art Water Retention Value (WRV) procedure. Saturation humidity procedure is easier and could be performed without dedicated equipment with respect to WRV.

Saturation humidity is correlated to torque/viscosity of the slurried ligno-cellulosic biomass, but it is related to not-slurried ligno-cellulosic biomass.

Saturation humidity was measured according to the following methodology:

An amount of 20 gr of sample on dry matter basis was inserted in a becker and water (up to 50 ml) was added in 2 ml aliquots every 1 h and hand shaken to allow the material adsorb the water. The procedure ends when water added is not absorbed into the material after the 1 h incubation and water drops are observed on the surface of the material. Measurements were performed at 25° C. The saturation humidity is calculated as the total amount of water absorbed into the material (initial moisture content plus the amount of water added), divided by the weight of the material on a dry basis.

The saturation humidity of samples prepared at different severity factor R₀₂ before and after fiber shives reduction is reported in FIG. 9. One of the effects of the disclosed fiber shives reduction treatment is to reduce the saturation humidity, and this result is also correlated to the decrease of torque/viscosity observed for ASR slurries with respect to BSR slurry. It is noted that in the prior art an increase of WRV (which is equivalent to saturation humidity) is usually related to micro-fibrillation of fibres, that is a mechanical treatment used to open up the fibres that consequently adsorb more water (see I. C. Hoeger et al., Cellulose (2013)20:807-818).

A similar concept is expressed in S. H. Lee et al., Bioresource Technology, 2010, 101, p. 9645-9649, and in S. H. Lee et al., Bioresource Technology, 2010, 101, p. 769-774, where a thermally treated biomass is subjected to a mechanical treatment by means on an extruder operated in condition to fibrillate the feedstock into submicron and/or nanoscale fibres, even if no WRV/saturation humidity measurements are presented.

Thereby, according to the prior art consideration, the fiber shives reduction treatment presently disclosed does not fibrillate the fibres.

Comparison of Torque of Slurried Thermally Treated Biomass after Fiber Shives Reduction and Thermally Treated Biomass Before Fiber Shives Reduction During Enzymatic Hydrolysis

To better demonstrate the importance of forming a low viscosity slurry from the thermally treated biomass after shives reduction without any added enzymes, a further sample was prepared, at the following conditions:

| | Soaking | | Steam explosion | | | | |
|---|---|---|---|---|---|---|---|
| Ligno- | Temper- | Time | Temper- | Time | | | |
| cellulosic | ature | (min- | ature | (min- | | | |
| biomass | (° C.) | utes) | (° C.) | utes) | R₀₁ | R₀₂ | R₀ |
| Wheat | 155 | 65 | 190 | 4 | 3.43 | 3.25 | 3.65 |
| straw | | | | | | | |

Fiber shives reduction step was performed by means of the extruder according to the process previously described.

Torque measurement experiments were run in two identical anchor impeller, herein referred to reactor A and reactor B, whose characteristics are here reported.
T (reactor diameter)=0.15 m−Z (reactor height)=0.30 m
jacket for heat exchange fluid all around the lateral surface and bottom, with a width of 4 cm;
hemi-spherical bottom;
cover with gasket and seal, with 5 openings (1 center hole for stirrer shaft; 4 side holes to 30 add materials or for sampling, that during the tests will be closed with caps).

The two reactors are fitted with two identical anchor agitators to give the following configurations:

D (“wingspan”)=0.136 m
S (blade width)=0.019 m
H (anchor height)=0.146 m
5 C (clearance, blade-wall distance)=0.007 m
Agitation was provided by Heidolph RZR 2102 control motors (power: 140 W).

With no material inserted, the no load torque at 23 rpm was 23 N cm. An amount of 800 gr of BSR material having a moisture content of 60% was inserted in reactor A and soaking liquid was added at a ratio of 1:0.67. The dry matter was progressively adjusted to reach a final dry matter of 15% by addition of water at the end of the experiment.

An amount of 800 gr of ASR material having a dry matter content of 40% was inserted in reactor B and soaking liquid was added at a ratio of 1:0.67. The dry matter was progressively adjusted to reach a final dry matter of 15% by addition of water at the end of the experiment.

Temperature in both reactors was 25° C.

The two mixtures were agitated at 23 rpm for 90 minutes with no enzymes added.

Viscosity reduction was then conducted in both reactors, at a temperature of 50° C.

pH was corrected to 5 by means of a KOH solution. Viscosity reduction was conducted by inserting Ctec3 enzymatic cocktail by Novozymes at a concentration of 4.5 gr of enzyme cocktail every 100 g gram of glucans contained in the BSR and ASR solid materials. Viscosity reduction was conducted for 48 hours under agitation.

Torque was recorded for all the experiment time. No load torque was subtracted by the measured torque. The torque of the mixture comprising the material before fiber shives reduction without enzymes was approximately constant at a value close to 110 N cm till the insertion of enzymes. Then torque value was found to decrease after enzyme addition as usually occurs during hydrolysis. The torque of the mixture comprising the material after fiber shives reduction was found to be very low and close to the torque value of the hydrolyzed stream even before enzymes addition. FIG. 10 reports torque values of the two slurries during the first 21 hours of mixing time. Torque values remained approximately constant after this period and for the remaining mixing time in both reactors. Time zero corresponds to the start of agitation. Arrows indicate enzymes addition in both reactors.

### Rheological and Viscosity Measurements

Different amounts of BSR and ASR of the sample having R₀₂=3.25 were added to water to prepare 600 ml slurry samples at different dry matter content on dry basis, ranging from 5 to 17%. The samples were agitated up to 15 minutes until reaching a visually well dispersed slurries.

Rheological measurements were performed using a RheolabQC at 25° C. Data were collected corresponding to a shear rate ranging from 0.01 to 100 s⁻¹ and at a slope of 6 Pt./dec. Table 4 reports the measured shear stress and viscosity values for ASR slurries having a dry matter of 5%, 7%, 9%, 11%. The viscosity is not constant and decreases with the increase of shear rate.

It was not possible to measure BSR slurries on RheolabQC at 25° C. even at a dry matter lower than 5% due to the high viscosity of the sample. This is a remarkable difference in the rheological properties of BSR and ASR slurries.

TABLE 4
Rheological parameters of ASR slurries having
a dry matter content of 5%, 7%, 9%, 11%.

| Shear | Shear Stress, Pa | | | | Viscosity, Pa · s | | | |
|---|---|---|---|---|---|---|---|---|
| Rate, | Dry matter | | | | | | | |
| 1/s | 5% | 7% | 9% | 11% | 5% | 7% | 9% | 11% |
| 0.10 | 0.72 | 0.69 | 1.11 | 18.10 | 7.2 | 6.90 | 11.1 | 181 |
| 0.15 | 0.68 | 0.82 | 0.71 | 20.30 | 4.66 | 5.60 | 4.84 | 138 |
| 0.22 | 0.63 | 1.26 | 0.62 | 23.60 | 2.9 | 5.87 | 2.9 | 110 |
| 0.32 | 0.62 | 1.84 | 0.94 | 27.70 | 1.97 | 5.82 | 2.97 | 87.7 |
| 0.46 | 1.14 | 1.63 | 1.33 | 35.10 | 2.47 | 3.50 | 2.87 | 75.7 |
| 0.68 | 0.96 | 1.53 | 0.64 | 47.70 | 1.41 | 2.25 | 0.932 | 70.1 |
| 1.00 | 1.17 | 1.16 | 1.19 | 58.10 | 1.17 | 1.16 | 1.19 | 58.2 |
| 1.47 | 0.81 | 0.67 | 1.01 | 43.20 | 0.553 | 0.45 | 0.687 | 29.3 |
| 2.15 | 0.67 | 1.00 | 1.35 | 10.70 | 0.31 | 0.47 | 0.627 | 4.94 |
| 3.16 | 1.36 | 1.77 | 1.00 | 27.10 | 0.429 | 0.56 | 0.317 | 8.61 |
| 4.64 | 0.54 | 1.11 | 1.78 | 18.50 | 0.117 | 0.24 | 0.383 | 3.97 |
| 6.81 | 0.77 | 1.33 | 1.96 | 36.60 | 0.113 | 0.20 | 0.288 | 5.36 |
| 10.00 | 0.74 | 1.56 | 3.23 | 25.30 | 0.074 | 0.16 | 0.323 | 2.53 |
| 14.70 | 1.09 | 1.64 | 4.35 | 28.20 | 0.074 | 0.11 | 0.296 | 1.92 |
| 21.50 | 1.16 | 1.89 | 5.61 | 26.20 | 0.053 | 0.09 | 0.26 | 1.21 |
| 31.60 | 1.61 | 2.05 | 5.05 | 22.40 | 0.050 | 0.06 | 0.16 | 0.70 |
| 46.40 | 0.73 | 2.75 | 4.63 | 24.90 | 0.015 | 0.06 | 0.099 | 0.53 |
| 68.10 | 0.37 | 2.45 | 5.84 | 24.30 | 0.005 | 0.04 | 0.085 | 0.35 |
| 100.00 | 0.44 | 2.62 | 4.36 | 21.60 | 0.004 | 0.03 | 0.043 | 0.21 |

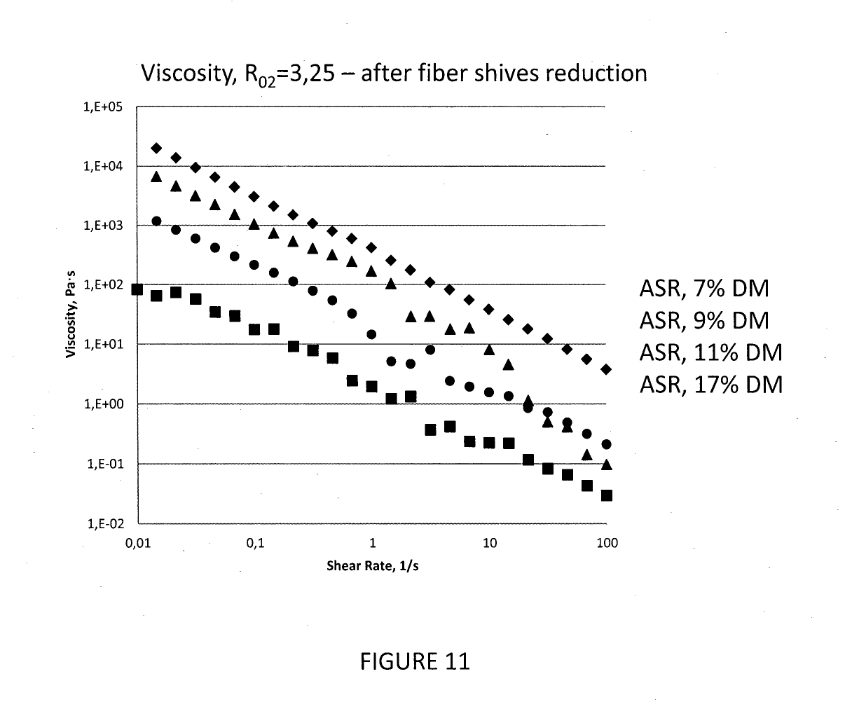

The viscosity of ASR slurries at 7%, 9%, 11% and 17% are reported in the graph of FIG. 11 on a bi-logarithmic scale. The vertical line in the graph indicates the shear rate value which was selected as the reference value for measuring the viscosity. In the context of the present disclosure, the described RheolabQC instrument procedure for viscosity measurement is the reference method for measuring the viscosity of a slurry.

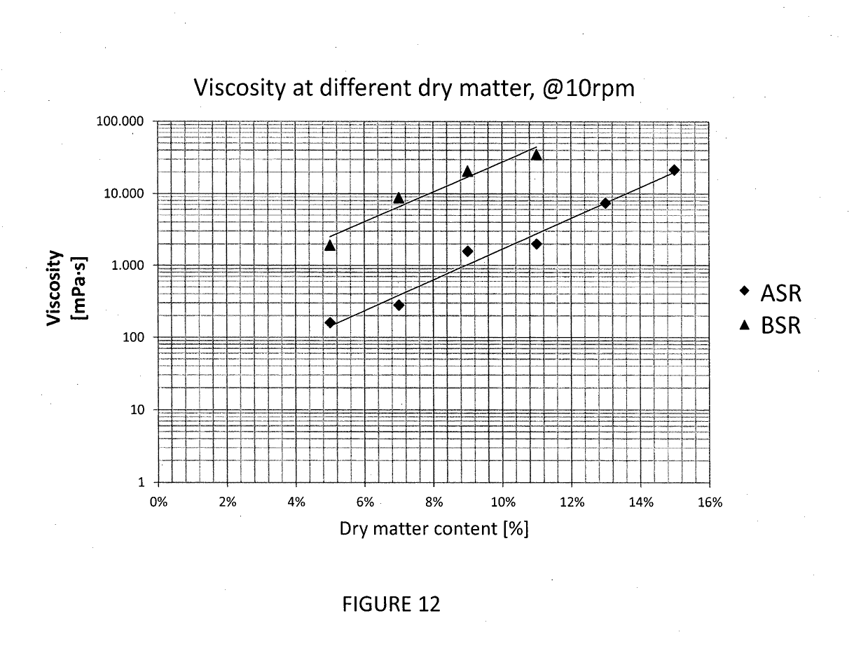

Viscosity measurements were performed on BSR and ASR slurry samples also using a Brookfield. RVDV-I Prime viscometer following the procedures reported by the producer. All the measurements were performed at 25° C. using a disc spindle #5 on a 600 ml sample. Data were collected starting from 1 rpm and increasing the rotation speed to 2.5, 5, 10, 20, 50 and 100 rpm. In FIG. 12 viscosities of BSR and ASR slurries collected at 10 rpm as a function of dry matter are shown. The graph highlights that the viscosity of the slurry prepared using ASR is about 90% less than that prepared using BSR.

### Hydrolyzate Preparation

The sample produced at R₀₂=3.25 was used for growing experiments.

The soaking liquid was subjected to a solid separation step to remove solids, by means of centrifugation and macro filtration (bag filter with filter size of 1 micron). Centrifugation was performed by means of a Alfa Laval CLARA 80 centrifuge at 8000 rpm. A clarified liquid was separated from suspended solids.

The clarified liquid was then subjected to a first nano-filtration step by means of a Alfa Laval 3.8″ equipment (membrane code NF3838/48), which splits the input stream into two streams, the retentate and the permeate. Nano-filtration was performed according to the following procedure.

Permeate flow stability was checked by means of flushing with de-mineral water, at the temperature of 50° C. and 10 bar. Flow rate of the permeate was measured. An amount of 1800 liter of clarified liquid were inserted in the feed tank. Before filtration, the system was flushed for 5 minutes, without pressure, in order to remove the water. The system was set at the operating conditions (pressure: 20 bar, temperature: 45° C.). Retentate stream was recycled in the feed tank and permeate stream was dumped. The test was run until the volume of liquid in the feed tank was reduced up to 50% of the initial soaked liquid volume, corresponding to 900 liters of permeate and 900 liters of retentate. The previous procedure produced a first nano-filtered retentate e a first nano-filtered permeated.

The first retentate liquid was diluted by adding a volume of water corresponding to 50% of its volume and subjected to a second first nano-filtration step, according to the same procedure used in the first nano-filtration step.

The second nano-filtration produced a second nano-filtered permeate and a purified liquid stream.

The steam exploded stream and the purified liquid stream were mixed in a bioreactor; water was added to reach a dry matter of 25%, then KOH was added to reach a pH of 5. Enzyme Ctec3 by Novozyme was added corresponding to a concentration of 30 mg/g of glucans and the mixture was hydrolyzed at 50° C. under continuous stirring for 48 hours.

Table 5 reports the compositions of soaking liquid, purified soaking liquid and steam exploded solid stream and hydrolyzate of wheat straw. For solid containing streams, i.e. steam exploded solid stream and hydrolyzate, composition is given in terms of percent weight of components, while for liquid streams, i.e., soaking liquid, purified soaking liquid, composition is given in terms of concentrations of components in the liquid. For hydrolyzate, which is composed by a solid fraction and a liquid fraction also concentrations of components in the liquid fraction are reported.

TABLE 5
Composition of wheat straw feedstock and streams

| | | | | | Hydro- |
|---|---|---|---|---|---|
| | Steam | | | Purified | lyzate |
| | exploded | Hydro- | Soaking | soaking | liquid |
| | solid stream | lyzate | liquid | liquid | fraction |
| | (% weight) | (% weight) | (g/l) | (g/l) | (g/l) |
| water | 59.98% | 77.32% | 629.8 | 972.6 | 827.3 |
| glucose | 0.00% | 4.12% | 0.0 | 0.6 | 48.6 |
| xylose | 0.15% | 2.56% | 1.5 | 1.7 | 30.2 |
| acetic acid | 0.10% | 0.24% | 1.0 | 2.3 | 2.8 |
| 5-HMF | 0.01% | 0.00% | 0.1 | 0.0 | 0.0 |
| furfural | 0.00% | 0.00% | 0.0 | 0.1 | 0.0 |
| glucolygomers | 0.25% | 0.30% | 2.6 | 7.4 | 3.5 |
| xylolygomers | 2.03% | 0.55% | 21.3 | 24.2 | 6.5 |
| soluble acetyls | 0.08% | 0.12% | 0.9 | 0.6 | 1.4 |
| unsoluble | 15.84% | 4.35% | NA | NA | NA |
| glucans | | | | | |
| unsoluble | 4.12% | 1.07% | NA | NA | NA |
| xylans | | | | | |
| unsoluble | 0.24% | 0.07% | NA | NA | NA |
| acetyls | | | | | |
| other solubles | 17.20% | 9.30% | 180.6 | 40.5 | 99.5 |
| and insolubles | | | | | |

### Yeast Growth

A commercial genetically modified yeast capable to ferment glucose and xylose was grown in batch configuration. In batch configuration, all the sugars are supplied to the cultivation environment before the inoculum of the yeast. Experiments presented are related to the growth of yeast on beet molasse in two different aerobic conditions as control experiments (CE1 and CE2) and to the growth of the yeast on two different cultivation environment based on ligno-cellulosic hydrolyzate WSH (WE1 and WE2).

The same growth protocol was applied in all the experiments, consisting of a inoculum (or pre-cultivation) phase on synthetic media followed by a growth phase in different cultivation environments.

### Inoculum Phase Protocol

Yeast was pre-cultured in a YPD medium with a standard procedure.

In Table 6 the composition of pre-cultivation environment is reported. Yeast extract, peptone and demineralized water are mixed and autoclaved at 121° C. for 30 minutes. Glucose solution is autoclaved at 110° C. for 20 minutes.

Nutrients were inserted in a shake flask (500 ml, operative volume 200 ml); a yeast inoculum starting concentration of 0.2 g/l was added to the flask and pre-cultured for 15 hours at 30° C., stirred at 150 rpm in micro-aeration condition obtained by sealing the flask with cotton lit. A pre-cultured yeast concentration of 2.5 g/l was obtained. Yeast concentrations were determined according to standard OD measurements at 700 nm.

TABLE 6
Composition of pre-cultivation medium

| | Concentration | Amount |
|---|---|---|
| Nutrients | (g/l) | (ml) |
| Yeast | 10 | |
| extract | | |
| Peptone | 20 | |
| H2Od | | 200 |
| (endvolume) | | |
| glucose | 20 | |

### Growth Phase Protocol

In Table 7 the composition of the cultivation environment in all the experiments is reported. The carbon source is varied and specified according to each experiment.

TABLE 7
Composition of the cultivation medium

| | Concentration | Amount |
|---|---|---|
| Nutrients | (g/l) | (ml) |
| Urea | 2.75 | |
| KH2PO4 | 3 | |
| H2Od | | 1870 |
| (endvolume) | | |
| Vitamine | 1 | |
| solution | | |
| Trace elements | 1 | |
| solution | | |
| Carbon source | 20-25 | |

The concentration of different vitamins in the vitamin solution is reported in Table 8 and the concentration of trace elements in the trace elements solution is reported in Table 9.

TABLE 8
Concentrations of vitamin solution

| | Concentration (g/l) |
|---|---|
| D-biotin | 0.05 |
| Ca-D-pantothenate | 1.00 |
| Nicotonic acid | 1.00 |
| Myo-inositol | 25.00 |
| Thiamine hydrochloride | 1.00 |
| Pyridoxal hydrochloride | 1.00 |
| p-aminobenzoic acid | 0.20 |

TABLE 9
Concentration of trace elements

| | Concentration (g/l) |
|---|---|
| Na2EDTA | 1.50 |
| ZnSO4•7H2O | 0.45 |
| MnCl2•2H2O | 0.10 |
| CoCl2•6H2O | 0.03 |
| CuSO4•5H2O | 0.03 |
| Na2MoO4•2H2O | 0.04 |
| CaCl2•2H2O | 0.45 |
| FeSO4•7H2O | 0.30 |
| H3BO3 | 0.10 |
| KI | 0.01 |

The carbon source, demineralized water and KH2PO4, according to the amounts reported in Table 7, were inserted in a bio-reactor (3.6 l, operative volume 2 l) and sterilized at 121° C. for 30 minutes; urea, vitamin solution and trace elements were added. A yeast amount coming from the inoculum, corresponding to an initial yeast concentration in the starting culture, was added to the bio-reactor.

Antifoam was added in a quantity sufficient to prevent foam, aeration was set and the temperature was set to 30° C. under agitation at 300 rpm. pH was adjusted to 5.

Yeast growth was performed in aerobic conditions of air flux of 1VVm and 18VVh. 1VVm is the air flux corresponding to an air volume equal to the cultivation medium volume per minute. 1VVm is the air flux corresponding to an air volume equal to the cultivation medium volume per hour. Considering the experimental setup, 1 VVm corresponds to 2 l/m and 18VVh correspond to 0.6 l/m.

Yeast amount and sugar concentration were measured at 0, 2, 4, 6, 7, 8, 10, 15, 24, 30 hours.

Culture was performed for 30 hours. Yeast growth performances were evaluated by considering the propagation factor, that is the ratio of the yeast amount at 24 hours to the starting yeast amount and the lag-time, defined as the time needed for first duplication, corresponding to propagation factor of 2.

A time of 24 h was selected for comparing the growth performance, considering that a higher time is inconvenient for industrial application.

In all the experiments, a relevant amount of the carbon source was converted to ethanol, due to the high carbon source concentration in the batch configuration, which promotes Crabtree effect. Because of this, it is meaningless to evaluate carbon-to-yeast conversion efficiency.

### Control Experiments CE1 and CE2

Two control experiments were defined, in which yeast was grown by using beet molasse as carbon source in aerobic conditions of air flux of 1 VVm and 18VVh respectively.

The composition of beet molasse in terms of sucrose, acetic acid and lactic acid is reported in Table 10. Other components comprises mainly reducing sugars and water, and minor amounts of calcium and ash. Reducing sugars are sugars that are not metabolized by yeast. The composition is in line with the mean composition of beet molasse, according to Chen, J. C. and C. C. Chou, 2003, Cane Sugar Handbook: A Manual for Cane Sugar Manufacturers and Their Chemists, John Wiley & Sons, New Jersey.

TABLE 10
Composition of beet molasse

| Molasses component | Weight % |
|---|---|
| sucrose | 37.4 |
| Lactic acid | 1.5 |
| Acetic acid | 0.6 |
| Other components | 60.5 |

Sucrose in the beet molasse was used as carbon source.

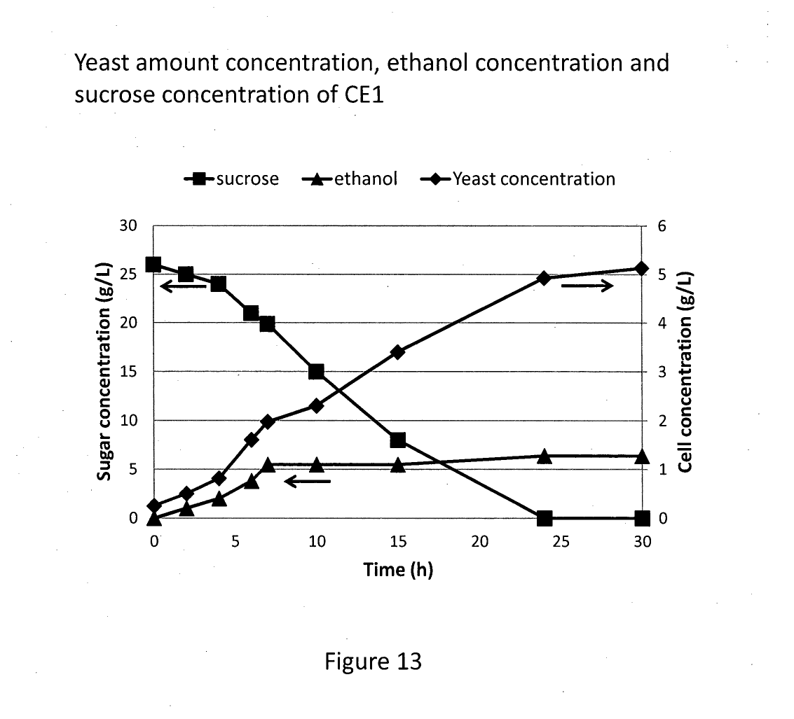

In CE1, performed at aeration condition of 1VVm at an initial sucrose concentration in the cultivation medium of 26 g/l and an initial yeast concentration of 0.247 g/l, a propagation factor of 19.93 at 24 h and a lag-phase of 2 h were obtained. In FIG. 13 the graph of yeast concentration, ethanol concentration and sucrose concentration of CE1 during the growth are reported as an example.

In CE2, performed at aeration condition of 18VVh at an initial sucrose concentration in the cultivation medium of 24.3 g/l and an initial yeast concentration of 0.161 g/l, a propagation factor of 19.99 at 24 h and a lag-phase slightly higher than 4 h were obtained.

### Working Experiments WE1-WE4.

In WE1 and WE2 glucose and xylose in the hydrolyzates of thermally treated wheat straw were used as carbon source. Sugars concentration was the sum of glucose and xylose concentration in the liquid fraction of hydrolyzate.

In WE1 the liquid fraction of hydrolyzates was separated from the solid fraction by means of filter paper followed by 0.22 μm membrane filtration. Only the liquid fraction, containing the soluble glucose and xylose, was used in growth experiments.

In WE2 the whole hydrolyzate, comprising both the liquid and the solid fractions, was used for growing the yeast.

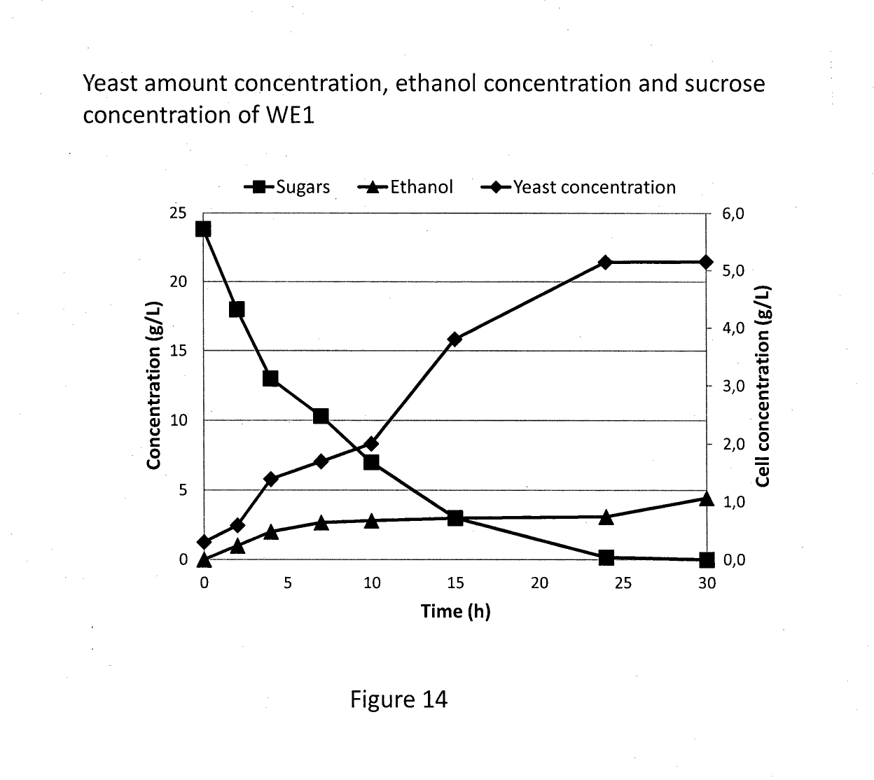

In WE1, performed at aeration condition of 1VVm at an initial sugars concentration in the cultivation medium in the of 23.39 g/l from liquid fraction of hydrolyzate, with an initial yeast concentration of 0.301 g/l, a propagation factor of 17.08 at 24 h and a lag-phase slightly higher than 2 h were obtained. In FIG. 14 the graph of yeast amount concentration, ethanol concentration and sugars concentration of WE1 during the growth is reported. Graphs relative to the growth of WE2 are similar, thereby they were omitted.

In WE2, performed at aeration condition of 1VVm, both the liquid and solid fractions of WSH hydrolyzate were used. The hydrolyzate was stirred before a homogeneous sample was taken. The growth was performed at an initial sugars concentration in the cultivation medium of 17.74 g/l from theliquid fraction, with an initial yeast concentration of 0.524 g/l, a propagation factor of 15.71 at 24 h and a lag-phase slightly less than 4 h were obtained.

The amount of yeast grown on the whole hydrolyzate was determined taking into account that yeast adheres completely on the solid fraction of the hydrolyzate and namely it is not detected in the liquid fraction. The variation of weight of dried solid fraction with respect to immediately after post-inoculum corresponds to the amount of grown yeast. The procedure was calibrated with OD measurements for reference.

In Table 11 experimental results are summarized. The reported lag-time is the time corresponding to the measured propagation factor closest to 2.

TABLE 11
Experimental results.

| | | Initial | | | | |
|---|---|---|---|---|---|---|
| | | carbon | Initial | | | |
| | | source | yeast | | Propa- | |
| | | concen- | concen- | | gation | Lag- |
| | Carbon | tration | tration | Aer- | factor@24 | time |
| | source | (g/l) | (g/l) | ation | h | (h) |
| CE1 | Beet | 26 | 0.247 | 1 VVm | 19.93 | 2 |
| | molasse | | | | | |
| CE2 | Beet | 24.3 | 0.161 | 18 VVh | 19.99 | 4 |
| | molasse | | | | | |
| WE1 | WSH liquid | 23.39 | 0.301 | 1 VVm | 17.04 | 2 |
| | fraction | | | | | |
| WE2 | WSH liquid | 17.74 | 0.524 | 1 VVm | 15.71 | 4 |
| | fraction | | | | | |

WE1 and WE2 demonstrate that the hydrolyzed composition produced according to the disclosed method can be used for growing yeast, obtaining performance comparable to those obtained by feeding the yeast with beet molasse. Even if propagation factor is slightly lower than in the case of molasse feed, great advantage is obtained in industrial applications, being the hydrolyzate directly produced in the industrial site at lower cost.

In Joao R. M. Almeida et al., “Screening of Saccharomyces cerevisiae strains with respect to anaerobic growth in non-detoxified lignocellulose hydrolyzate”, Biores. Tech. 100 (2009), 3674, anaerobic growth of 12 Saccharomyces cerevisiae strains were grown in three different hydrolyzates. The composition of barley straw hydrolysate, two-step dilute-acid spruce hydrolyzate and wheat straw were in g/l, respectively, 1.1, 42.9, 6.4 glucose, 1.0, 24.4, 0.6 mannose, 0.5, 7.7, 1.1 galactose, 3.5, 10.4, 35.4 xylose, 5.6, 6.2, 4.01 acetic acid, 0.9, 3.6, 0.6 HMF and 3.1, 2.1, 1.8 furfural. Growth was measured after approximately 45 h of incubation in hydrolysate concentrations up to 50%, 60% and 70% for barley straw, spruce and wheat straw hydrolysate, respectively (FIG. 1 of the paper). The authors point out that growth was not detected in any hydrolysate at 100%, even after more than 100 h of incubation. The toxicity of the hydrolysates correlated with the duration of yeast lag phase, which in the highest hydrolysate concentration where growth was detected was approximately 35 h in barley straw, the most inhibitory hydrolysate, 25 h in spruce and 15 h in wheat straw, the least inhibitory one. Moreover, from FIG. 5 it is evident that in the case of 70% of wheat straw hydrolyzate a maximum propagation factor of approximately 3 was obtained after approximately 24 hours and a maximum propagation factor of 5 was obtained after approximately 35 hours.

Experimental results clearly highlight the improvement of the disclosed method for growing yeast with respect to state of the art.

1-25. (canceled)

26. A process for growing a microbial organism comprising the steps of:
a. thermally treating a ligno-cellulosic biomass feedstock to create a thermally treated ligno-cellulosic biomass, said thermally treated ligno-cellulosic biomass comprising xylans, glucans and lignin;
b. dispersing an amount of the thermally treated ligno-cellulosic biomass into an amount of a carrier liquid to create a slurry;
c. contacting the slurry with an enzyme under hydrolysis conditions of a carbohydrate component of the slurry to produce a hydrolyzed composition comprising simple sugar or sugars derived from the xylans and glucans of the thermally treated biomass, wherein the simple sugar or sugars can be metabolized by the microbial organism;
d. cultivating the microbial organism in a cultivation environment comprising at least a portion of the hydrolyzed composition under conditions and for a cultivation time sufficient to grow the microbial organism.

27. The process of claim 26, wherein the thermally treated ligno-cellulosic biomass is in physical forms of at least fibres, fines and fiber shives, wherein:
i. the fibres each have a width of 75 μm or less, and a fibre length greater than or equal to 200 μm,
ii. the fines each have a width of 75 μm or less, and a fine length of less than 200 μm,
iii. the fiber shives each have a shive width greater than 75 μm with a first portion of the fiber shives each having a shive length less than 737 μm and a second portion of the fiber shives each having a shive length greater than or equal to 737 μm; and
wherein the process further comprises the step of reducing the fiber shives of the thermally treated biomass, wherein the percent area of fiber shives having a shive length greater than or equal to 737 μm relative to the total area of fiber shives, fibres and fines of the thermally treated ligno-cellulosic biomass after fiber shives reduction is less than the percent area of fiber shives having a shive length greater than or equal to 737 μm relative to the total area of fiber shives, fibres and fines of the thermally treated ligno-cellulosic biomass before fiber shives reduction, wherein the total area of fiber shives, fibres and fines is measured by automated optical analysis.

28. The process according to claim 27, wherein a part of the fiber shives reduction is done by separating at least a portion of the fiber shives having a shive length greater than or equal to 737 μm from the thermally treated ligno-cellulosic biomass.

29. The process of claim 27, wherein a part of the fiber shives reduction is done by converting at least a portion of the fiber shives having a shive length greater than or equal to 737 μm in the thermally treated ligno-cellulosic biomass to fibres or fines.

30. The process of claim 27, wherein at least a part of the fiber shives reduction step is done by applying a work in a form of mechanical forces to the thermally treated ligno-cellulosic biomass, and all the work done by all the forms of mechanical forces on the thermally treated ligno-cellulosic biomass is less than 500 Wh/Kg per kg of the thermally treated ligno-cellulosic biomass on a dry basis.

31. The process of claim 30, wherein all the work done by all the forms of mechanical forces on the thermally treated ligno-cellulosic biomass is less than a value selected from the group consisting of 400 Wh/Kg, 300 Wh/Kg, 200 Wh/Kg, 100 Wh/Kg, per kg of the thermally treated ligno-cellulosic biomass on a dry basis.

32. The process of claim 27, wherein the percent area of the fiber shives having a shive length greater than or equal to 737 μm relative to the total area of fiber shives, fibres and fines of the thermally treated ligno-cellulosic biomass after fiber shives reduction is less than a value selected from the group consisting of 1%, 0.5%, 0.25%, 0.2% and 0.1%.

33. The process of claim 27, wherein the slurry has a viscosity less than a value selected from the group consisting of 0.1 Pa s, 0.3 Pa s, 0.5 Pa s, 0.7 Pa s, 0.9 Pa s, 1.0 Pa s, 1.5 Pa s, 2.0 Pa s, 2.5 Pa s, 3.0 Pa s, 4 Pa s, 5 Pa s, 7 Pa s, 9 Pa s, 10 Pa s, wherein the viscosity is measured at 25° C., at a shear rate of 10 s-1 and at a dry matter of 7% by weight.

34. The process of claim 27, wherein the dry matter of the slurry by weight is higher than a value selected from the group consisting of 5%, 7%, 8%, 10%, 12%, 15%, 18%, 20%, 25%, 30%, 35%, 40%.

35. The process of claim 26, wherein the slurry does not contain ionic groups derived from added mineral acids, mineral bases, organic acids, or organic bases.

36. The process of claim 26, wherein the thermal treatment of the ligno-cellulosic biomass feedstock comprises the step of steam exploding the ligno-cellulosic biomass feedstock to create the thermally pre-treated ligno-cellulosic biomass.

37. The process of claim 36, wherein the steam explosion step is preceded by the steps of:
a. soaking the ligno-cellulosic biomass feedstock in vapor or liquid water or mixture thereof in the temperature range of 100 to 210° C. for 1 minute to 24 hours to create a soaked ligno-cellulosic biomass feedstock containing a solid content and a liquid content;
b. separating at least a portion of the liquid content from the soaked ligno-cellulosic biomass feedstock to create a solid stream and a liquid stream, wherein the solid stream comprises the ligno-cellulosic biomass feedstock, which has been soaked.

38. The process of claim 37, wherein the carrier liquid comprises at least a portion of the liquid stream.

39. The process of claim 26, wherein the conversion of the ligno-cellulosic biomass feedstock to the slurry is conducted without the addition of a hydrolysis catalyst.

40. The process of claim 26, wherein the hydrolyzed composition comprises acetic acid and the ratio of the amount of acetic acid to the total amount of simple sugar or sugars is less than a value selected from the group consisting of 0.15, 0.10, 0.05, 0.02 and 0.01.

41. The process of claim 26, wherein the hydrolyzed composition comprises furfural and the ratio of the amount of furfural to the total amount of simple sugar or sugars in the hydrolyzed composition is less than a value selected from the group consisting of 0.01, 0.005, 0.001, 0.0005, and 0.0003.

42. The process of claim 26, wherein the hydrolyzed composition comprises 5HMF and the ratio of the amount of 5HMF to the total amount of the simple sugar or sugars in the hydrolyzed composition is less than a value selected from the group consisting of 0.02, 0.02, 0.005, 0.001, and 0.0005.

43. The process of claim 26, wherein the cultivation of the microbial organism is done without added simple sugar or sugars to the cultivation environment.

44. The process of claim 26, wherein the cultivation of the microbial organism is done with added simple sugar or sugars, and the percent ratio of the amount of added simple sugar or sugars to the total amount of simple sugar or sugars of the hydrolyzed composition is less than a value selected from the group consisting of 30%, 20%, 10%, 5.0%, and 2.0%.

45. The process of claim 26, wherein the cultivation time is less than a value selected from the group consisting of 36 hours, 24 hours, 18 hours, 12 hours and 6 hours.

46. The process of claim 26, wherein the cultivation of the microbial organism is performed in aerobic condition at an air flow which is less than a value selected from the group consisting of 1 VVm, 10 VVh, 5 VVh, 1 VVh, 0.5 VVh, 0.1 VVh, and 0.05 VVh.

47. The process of claim 26, wherein the microbial organism is a non-naturally occurring microbial organism.

48. The process of claim 26, wherein the microbial organism is a yeast.

49. The process of claim 48, wherein the yeast is selected from the group consisting of Saccharomyces, Zygosaccharomyces, Candida, Hansenula, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, and Schwanniomyces.

50. The process of claim 26, wherein the microbial organism is a bacterium.
